(19) United States
(12) Patent Application Publication
(10) Pub. No.: US 2019/0346452 A1
(43) Pub. Date: Nov. 14, 2019

(54) METHODS AND COMPOSITIONS FOR PROVIDING PREECLAMPSIA ASSESSMENT

(71) Applicant: LDX Prognostics Limited Co., Jiangsu (CN)

(72) Inventor: Wenkai XIANG, Belmont, CA (US)

(21) Appl. No.: 16/301,701

(22) PCT Filed: May 17, 2016
(86) PCT No.: PCT/CN2016/082314

§ 371 Date: Nov. 14, 2018

(51) Int. Cl. G01N 33/68

(52) U.S. Cl. CPC G01N 33/689; G01N 2333/4706; G01N 2800/368; G01N 2333/71

(57) ABSTRACT

Disclosed is a method for providing a preeclampsia assessment for example diagnosis of preeclampsia by evaluating a panel of preeclampsia markers including Activin A. A kit used in the preeclampsia assessment is also disclosed.

### CROSS-REFERENCE TO RELATED APPLICATION

This application is a U.S. National Stage Application under 35 U.S.C. § 371 of International Application No. PCT/CN2016/082314, filed May 17, 2016, the content of which is incorporated of the present disclosure by reference.

### BACKGROUND OF THE INVENTION

Preeclampsia is a serious multisystem complication of pregnancy with adverse effects for mothers and babies. The incidence of the disorder is around 5-8% of all pregnancies in the U.S. and worldwide, and the disorder is responsible for 18% of all maternal deaths in the U.S. The causes and pathogenesis of preeclampsia remain uncertain, and the diagnosis relies on nonspecific laboratory and clinical signs and symptoms that occur late in the disease process, sometimes making the diagnosis and clinical management decisions difficult. Earlier and more reliable disease diagnosing, prognosing and monitoring will lead to more timely and personalized preeclampsia treatments and significantly advance our understanding of preeclampsia pathogenesis.

### SUMMARY OF THE INVENTION

Preeclampsia markers, preeclampsia marker panels, and methods for obtaining a preeclampsia marker level representation for a sample are provided. These compositions and methods find use in a number of applications, including, for example, diagnosing preeclampsia, prognosing a preeclampsia, monitoring a subject with preeclampsia, and determining a treatment for preeclampsia. In addition, systems, devices and kits thereof that find use in practicing the subject methods are provided.

In some aspects of the invention, a panel of preeclampsia markers is provided, the panel comprising one or more preeclampsia markers selected from the group consisting of inhibin beta A (Activin A), endoglin (ENG), endothelial protein C receptor (EPCR), soluble fms-like tyrosine kinase-1 (sFlt-1), and placenta growth factor (PIGF).

In some aspects of the invention, a method is provided for providing a preeclampsia marker level representation for a subject. In some embodiments, the method comprises evaluating a panel of preeclampsia markers in a blood sample from a subject to determine the level of each preeclampsia marker in the blood sample; and obtaining the preeclampsia marker level representation based on the level of each preeclampsia marker in the panel. In some embodiments, the panel comprises inhibin beta A (Activin A). In some embodiments, the panel comprises inhibin beta A (Activin A) and placenta growth factor (PIGF). In some embodiments, the panel further comprises one or more preeclampsia markers selected from the group consisting of endoglin (ENG), endothelial protein C receptor (EPCR), and soluble fms-like tyrosine kinase-1 (sFlt-1). In some embodiments, the panel comprises one or more preeclampsia markers selected from the group consisting of inhibin beta A, endoglin (ENG), endothelial protein C receptor (EPCR), soluble fms-like tyrosine kinase-1 (sFlt-1) and placenta growth factor (PIGF). In some embodiments, the method further comprises providing a report of the preeclampsia marker level representation. In certain embodiments, the preeclampsia marker representation is a preeclampsia score.

In some aspects of the invention, a method is provided for providing a preeclampsia assessment for a subject. In some embodiments, the preeclampsia assessment is a diagnosis of preeclampsia. In some embodiments, the method comprises obtaining a preeclampsia marker level representation for a sample from a subject, e.g. as described above or elsewhere herein, and providing a preeclampsia diagnosis for the subject based on the preeclampsia marker level representation. In some embodiments, the method further comprises comparing the preeclampsia marker level representation to a preeclampsia phenotype determination element, and providing a preeclampsia diagnosis for the subject based on the comparison. In some embodiments, the subject has symptoms of preeclampsia. In other embodiments, the subject is asymptomatic for preeclampsia. In some embodiments, the subject has one or more risk factors associated with preeclampsia. In other embodiments, the subject has no risk factors associated with preeclampsia. In some embodiments, the sample is collected at 16 or more weeks of gestation. In certain embodiments, the sample is collected at 34 or more weeks of gestation.

In one embodiment, the methods of the present disclosure do not include measurement of expression levels of ADAM12 and/or PAPPA2. In one embodiment, the methods do not include measurement of expression levels of FSTL3, APLN, LEP, INHA, PIK3CB, SLC2A1, CRH, HSD17B1, SIGLEC6, PVRL4, HEXB, ID RAP, MFAP5, HTRA1, EB13, HTRA4.

In one embodiment, the methods do not include measurement of the expression level of ADAM12. In one embodiment, the methods do not include measurement of the expression level of PAPPA2.

In one embodiment, the methods do not include measurement of the expression level of FSTL3. In one embodiment, the methods do not include measurement of the expression level of APLN. In one embodiment, the methods do not include measurement of the expression level of LEP. In one embodiment, the methods do not include measurement of the expression level of INHA. In one embodiment, the methods do not include measurement of the expression level of PIK3CB. In one embodiment, the methods do not include measurement of the expression level of SLC2A1. In one embodiment, the methods do not include measurement of the expression level of CRH. In one embodiment, the methods do not include measurement of the expression level of HSD17B1. In one embodiment, the methods do not include measurement of the expression level of SIGLEC6. In one embodiment, the methods do not include measurement of the expression level of PVRL4. In one embodiment, the methods do not include measurement of the expression level of HEXB. In one embodiment, the methods do not include measurement of the expression level of IL1 RAP. In one embodiment, the methods do not include measurement of the expression level of MFAP5. In one embodiment, the methods do not include measurement of the expression level of HTRA1. In one embodiment, the methods do not include measurement of the expression level of EB13. In one embodiment, the methods do not include measurement of the expression level of HTRA4.

The methods may be particularly suitable for certain pregnant women, such as those that have history of preeclampsia, have obesity, have babies less than two years or more than 10 years apart, are older than 40, have history of certain conditions including chronic high blood pressure, migraine headaches, type 1 or type 2 diabetes, kidney disease, a tendency to develop blood clots, or lupus.

Once the diagnosis of preeclampsia is determined, the woman can be subject to a procedure that helps ameliorate the preeclampsia. Examples of such procedures include, without limitation, medications to lower blood pressure, use of corticosteroids, anticonvulsant medication such as magnesium sulfate, bed rest, and consideration of delivery if the diagnosis was made at or after 37 gestational weeks.

In some aspects of the invention, a panel of preeclampsia markers is provided comprising inhibin beta A (Activin A). In some embodiments, the panel of preeclampsia markers comprises inhibin beta A (Activin A) and placenta growth factor (PIGF). In some embodiments, the panel of preeclampsia markers consists of inhibin beta A (Activin A) and placenta growth factor (PIGF).

In some aspects of the invention, a kit is provided for making a preeclampsia assessment for a sample. In some embodiments, the preeclampsia assessment is a preeclampsia diagnosis. In some embodiments, the kit comprises one or more detection elements for measuring the amount of marker in a sample for a panel of preeclampsia markers comprising inhibin beta A (Activin A). In some embodiments, the kit comprises one or more detection elements for measuring the amount of marker in a sample for a panel of preeclampsia markers comprising inhibin beta A (Activin A) and placenta growth factor (PIGF). In some embodiments, the kit comprises one or more detection elements for measuring the amount of marker in a sample for a panel of preeclampsia markers further comprising one or more markers selected from the group consisting of endoglin (ENG), endothelial protein C receptor (EPCR), and soluble fms-like tyrosine kinase-1 (sFlt-1). In some embodiments, the kit further comprises a preeclampsia phenotype determination element. In some embodiments, the kit comprises one or more detection elements for measuring the amount of marker in a sample for a panel of preeclampsia markers comprising one or more markers selected from the group consisting of inhibin beta A (Activin A), endoglin (ENG), endothelial protein C receptor (EPCR), soluble fms-like tyrosine kinase-1 (sFlt-1), and placenta growth factor (PIGF); and a preeclampsia phenotype determination element. In some embodiments, the one or more detection elements detect the level of marker polypeptides in the sample.

In some aspects of the invention, use of the kit as described above in the preparation of a composition for making a preeclampsia assessment for a sample is provided. The preeclampsia assessment includes e.g. diagnosing, prognosing, monitoring, and/or treating preeclampsia in a subject.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Study outline of the multi-‘omics’, based discovery and validation of PE biomarkers.

FIG. 2. Identification of PE biomarkers using a combination of meta-analysis, protein atlas analysis, and human orthologues analysis.

FIG. 3. Expression comparative analysis of PE biomarkers (PE versus controls). Forest plot summarizes the results of placenta mRNA expression meta analysis, and maternal serum analyte abundance quantification at different early and late gestational age weeks. Line plot represents 95% confidence interval.

FIG. 4. A: Boxplot display and scatter plot of biomarker distribution for Activin A at different gestational age weeks at blood sample collection in PE and control groups. Horizontal box boundaries and midline denote sample quartiles. B: Scatter plots of biomarker distribution for Activin A as a function of gestational age weeks at blood sample collection (Top), delivery (Bottom), and the gap in between (Middle).

FIG. 5. A: Boxplot display and scatter plot of biomarker distribution for ENG at different gestational age weeks at blood sample collection in PE and control groups. Horizontal box boundaries and midline denote sample quartiles. B: Scatter plots of biomarker distribution for ENG as a function of gestational age weeks at blood sample collection (Top), delivery (Bottom), and the gap in between (Middle).

FIG. 6. A: Boxplot display and scatter plot of biomarker distribution for EPCR at different gestational age weeks at blood sample collection in PE and control groups. Horizontal box boundaries and midline denote sample quartiles. B: Scatter plots of biomarker distribution for EPCR as a function of gestational age weeks at blood sample collection (Top), delivery (Bottom), and the gap in between (Middle).

FIG. 7. A: Boxplot display and scatter plot of biomarker distribution for PIGF at different gestational age weeks at blood sample collection in PE and control groups. Horizontal box boundaries and midline denote sample quartiles. B: Scatter plots of biomarker distribution for PIGF as a function of gestational age weeks at blood sample collection (Top), delivery (Bottom), and the gap in between (Middle).

FIG. 8. A: Boxplot display and scatter plot of biomarker distribution for sFlt-1 at different gestational age weeks at blood sample collection in PE and control groups. Horizontal box boundaries and midline denote sample quartiles. B: Scatter plots of biomarker distribution for sFlt-1 A as a function of gestational age weeks at blood sample collection (Top), delivery (Bottom), and the gap in between (Middle).

FIG. 9. Biomarker panel scores were plotted as a function of the gestational weeks at blood sample collection. * Loess curves were fitted to represent the overall trend of biomarker scoring as a function of gestational age.

FIG. 10. A: Biomarker panel scores (Top) and the associated ROC curves (Bottom) were plotted as a function of the gestational weeks at blood sample collection. The scores were produced by a random forest algorithm developed (A) with a panel of all five validated biomarkers as well as the gestational weeks, and (B) with a panel of all five validated biomarkers.

### DETAILED DESCRIPTION OF THE INVENTION

Preeclampsia markers, preeclampsia marker panels, and methods for obtaining a preeclampsia marker level representation for a sample are provided. These compositions and methods find use in a number of applications, including, for example, diagnosing preeclampsia, prognosing a preeclampsia, monitoring a subject with preeclampsia, and determining a treatment for preeclampsia. In addition, systems, devices and kits thereof that find use in practicing the subject methods are provided. These and other objects, advantages, and features of the invention will become apparent to those persons skilled in the art upon reading the details of the compositions and methods as more fully described below.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As summarized above, aspects of the subject invention include methods, compositions, systems and kits that find use in providing a preeclampsia assessment, e.g. diagnosing, prognosing, monitoring, and/or treating preeclampsia in a subject. By “preeclampsia” or “pre-eclampsia” it is meant a multisystem complication of pregnancy that may be accompanied by one or more of high blood pressure, proteinuria, swelling of the hands and face/eyes (edema), sudden weight gain, higher-than-normal liver enzymes, and thrombocytopenia. Preeclampsia typically occurs in the third trimester of pregnancy, but in severe cases, the disorder occur in the second trimester, e.g., after about the 22^{nd} week of pregnancy. If unaddressed, preeclampsia can lead to eclampsia, i.e. seizures that are not related to a preexisting brain condition. By “diagnosing” a preeclampsia or “providing a preeclampsia diagnosis,” it is generally meant providing a preeclampsia determination, e.g. a determination as to whether a subject (e.g. a subject that has clinical symptoms of preeclampsia, a subject that is asymptomatic for preeclampsia but has risk factors associated with preeclampsia, a subject that is asymptomatic for preeclampsia and has no risk factors associated with preeclampsia) is presently affected by preeclampsia; a classification of the subject's preeclampsia into a subtype of the disease or disorder; a determination of the severity of preeclampsia; and the like. By “prognosing” a preeclampsia, or “providing a preeclampsia prognosis,” it is generally meant providing a preeclampsia prediction, e.g. a prediction of a subject's susceptibility, or risk, of developing preeclampsia; a prediction of the course of disease progression and/or disease outcome, e.g. expected onset of the preeclampsia, expected duration of the preeclampsia, expectations as to whether the preeclampsia will develop into eclampsia, etc.; a prediction of a subject's responsiveness to treatment for the preeclampsia, e.g., positive response, a negative response, no response at all; and the like. By “monitoring” a preeclampsia, it is generally meant monitoring a subject's condition, e.g. to inform a preeclampsia diagnosis, to inform a preeclampsia prognosis, to provide information as to the effect or efficacy of a preeclampsia treatment, and the like. By “treating” a preeclampsia it is meant prescribing or providing any treatment of a preeclampsia in a mammal, and includes: (a) preventing the preeclampsia from occurring in a subject which may be predisposed to preeclampsia but has not yet been diagnosed as having it; (b) inhibiting the preeclampsia, i.e., arresting its development; or (c) relieving the preeclampsia, i.e., causing regression of the preeclampsia.

In describing the subject invention, compositions useful for providing a preeclampsia assessment will be described first, followed by methods, systems and kits for their use.

### Preeclampsia Markers and Panels

In some aspects of the invention, preeclampsia markers and panels of preeclampsia markers are provided. By a “preeclampsia marker” it is meant a molecular entity whose representation in a sample is associated with a preeclampsia phenotype. For example, a preeclampsia marker may be differentially represented, i.e. represented at a different level, in a sample from an individual that will develop or has developed preeclampsia as compared to a healthy individual. In some instances, an elevated level of marker is associated with the preeclampsia phenotype. For example, the concentration of marker in a sample may be 1.5-fold, 2-fold, 2.5-fold, 3-fold, 4-fold, 5-fold, 7.5-fold, 10-fold, or greater in a sample associated with the preeclampsia phenotype than in a sample not associated with the preeclampsia phenotype. In other instances, a reduced level of marker is associated with the preeclampsia phenotype. For example, the concentration of marker in a sample may be 10% less, 20% less, 30% less, 40% less, 50% less or more in a sample associated with the preeclampsia phenotype than in a sample not associated with the preeclampsia phenotype.

Preeclampsia markers may include proteins associated with preeclampsia and their corresponding genetic sequences, i.e. mRNA, DNA, etc. By a “gene” or “recombinant gene” it is meant a nucleic acid comprising an open reading frame that encodes for the protein.

The boundaries of a coding sequence are determined by a start codon at the 5′ (amino) terminus and a translation stop codon at the 3′ (carboxy) terminus. A transcription termination sequence may be located 3′ to the coding sequence. In addition, a gene may optionally include its natural promoter (i.e., the promoter with which the exons and introns of the gene are operably linked in a non-recombinant cell, i.e., a naturally occurring cell), and associated regulatory sequences, and may or may not have sequences upstream of the AUG start site, and may or may not include untranslated leader sequences, signal sequences, downstream untranslated sequences, transcriptional start and stop sequences, polyadenylation signals, translational start and stop sequences, ribosome binding sites, and the like.

As demonstrated in the examples of the present disclosure, the inventors have identified a number of molecular entities that are associated with preeclampsia and that find use in combination (i.e. as a panel) in providing a preeclampsia assessment, e.g. diagnosing preeclampsia, prognosing a preeclampsia, monitoring a subject with preeclampsia, determining a treatment for a subject affected with preeclampsia, and the like. These include, but are not limited to, inhibin beta A (Activin A, Genbank Accession No. NM_002192); Endoglin (ENG, Genbank Accession Nos. NM_000118, NM_001114753, NM_001278138), Protein C Receptor, Endothelial (EPCR, Genbank Accession No. NM_006404); Placental Growth Factor (PIGF, Genbank Accession Nos. NM_001207012 NM_002632 NM_001293643); and Soluble fms-like tyrosine kinase-1 (sFlt-1, Genbank Accession Nos. NM_001160030, NM_001160031, NM_002019, NM_001159920).

As mentioned above, also provided herein are preeclampsia panels. By a “panel” of preeclampsia markers it is meant two or more preeclampsia markers, e.g. 3 or more, 4 or more, or 5 or more markers, whose levels, when considered in combination, find use in providing a preeclampsia assessment, e.g. making a preeclampsia diagnosis, prognosis, monitoring, and/or treatment. Of particular interest are panels that comprise the preeclampsia marker Activin A, ENF, EPCR, and PIGF. For example, in some embodiments, the preeclampsia panel may comprise Activin A, PIGF, and one of more of ENG and EPCR. e.g. it may comprise Activin A and PIGF; Activin A, ENG, and PIGF; Activin A, EPCR, and PIGF; or Activin A, ENG, EPCR, and PIGF.

In some instances, other preeclampsia markers known in the art may be included in the subject preeclampsia panels, e.g. soluble vascular endothelial growth factor/vascular permeability factor receptor (VEGF-R1, also known as FMS-like tyrosine kinase 1 or sFlt-1; Genbank Accession Nos. NM_001159920.1 (isoform 2), NM_001160030.1 (isoform 3), and NM_001160031.1 (isoform 4)); and placental growth factor (PIGF, Genbank Accession Nos. NM_002632.5 (isoform 1) and NM 001207012.1 (isoform 2)) (Verlohren et al. (2010) Amer Journal of Obstetrics and Gynecology 161: e1-e11). Thus, for example, the preeclampsia panel may comprise sFlt-1, PIGF, and one or more of Activin A, ENG, and EPCR. e.g. it may comprise sFlt-1 and PIGF; sFlt-1, Activin A, and PIGF; sFlt-1, Activin A, ENG, and PIGF; sFlt-1, Activin A, EPCR, and PIGF; sFlt-1, ENG, and PIGF; sFlt-1, EPCR, and PIGF; sFlt-1, ENG, EPCR, and PIGF; or sFlt-1, Activin A, ENG, EPCR, and PIGF.

Other combinations of preeclampsia markers that find use as preeclampsia panels in the subject methods may be readily identified by the ordinarily skilled artisan using any convenient statistical methodology, e.g. as known in the art or described in the working examples herein. For example, the panel of analytes may be selected by combining genetic algorithm (GA) and all paired (AP) support vector machine (SVM) methods for preeclampsia classification analysis. Predictive features are automatically determined, e.g. through iterative GA/SVM, leading to very compact sets of non-redundant preeclampsia-relevant analytes with the optimal classification performance. While different classifier sets will typically harbor only modest overlapping gene features, they will have similar levels of accuracy in providing a preeclampsia assessment to those described above and in the working examples herein.

### Methods

In some aspects of the invention, methods are provided for obtaining a preeclampsia marker level representation for a subject. By a preeclampsia marker level representation, it is meant a representation of the levels of one or more of the subject preeclampsia marker(s), e.g. a panel of preeclampsia markers, in a biological sample from a subject. The term “biological sample” encompasses a variety of sample types obtained from an organism and can be used in a diagnostic, prognostic, or monitoring assay. The term encompasses blood and other liquid samples of biological origin or cells derived therefrom and the progeny thereof. The term encompasses samples that have been manipulated in any way after their procurement, such as by treatment with reagents, solubilization, or enrichment for certain components. The term encompasses a clinical sample, and also includes cell supernatants, cell lysates, serum, plasma, biological fluids, and tissue samples. Clinical samples for use in the methods of the invention may be obtained from a variety of sources, particularly blood samples.

Sample sources of particular interest include blood samples or preparations thereof, e.g., whole blood, or serum or plasma, and urine. A sample volume of blood, serum, or urine between about 2 μl to about 2,000 μl is typically sufficient for determining the level of a preeclampsia gene product. Generally, the sample volume will range from about 10 μl to about 1,750 μl, from about 20 μl to about 1,500 μl, from about 40 μl to about 1,250 μl, from about 60 μl to about 1,000 μl, from about 100 μl to about 900 μl, from about 200 μl to about 800 μl, from about 400 μl to about 600 μl. In many embodiments, a suitable initial source for the human sample is a blood sample. As such, the sample employed in the subject assays is generally a blood-derived sample. The blood derived sample may be derived from whole blood or a fraction thereof, e.g., serum, plasma, etc., where in some embodiments the sample is derived from blood, allowed to clot, and the serum separated and collected to be used to assay.

In some embodiments the sample is a serum or serum-derived sample. Any convenient methodology for producing a fluid serum sample may be employed. In many embodiments, the method employs drawing venous blood by skin puncture (e.g., finger stick, venipuncture) into a clotting or serum separator tube, allowing the blood to clot, and centrifuging the serum away from the clotted blood. The serum is then collected and stored until assayed. Once the patient derived sample is obtained, the sample is assayed to determine the level of preeclampsia marker(s).

The subject sample is typically obtained from the individual during the second or third trimester of gestation. By “gestation” it is meant the duration of pregnancy in a mammal, i.e. the time interval of development from fertilization until birth, plus two weeks, i.e. to the first day of the last menstrual period. By the second or third trimester, it is meant the second or third portions of gestation, each segment being 3 months long. Thus, for example, by the “first trimester” is meant from the first day of the last menstrual period through the 13th week of gestation; by the “second trimester” it is meant from the 14th through 27th week of gestation; and by the “third trimester” it is meant from the 28th week through birth, i.e. 38-42 weeks of gestation. Put another way, a subject sample may be obtained at about weeks 14 through 42 of gestation, at about weeks 18 through 42 of gestation, at about weeks 20 through 42 of gestation, at about weeks 24 through 42 of gestation, at about weeks 30 through 42 of gestation, at about weeks 34 through 42 of gestation, at about weeks 38 through 42 of gestation. Thus, in some embodiments, the subject sample may be obtained early in gestation, e.g. at week 14 or more of gestation, e.g. at week 14,15,16,17,18,19, 20, 21,22, or 23 or more of gestation, more often at week 24, 25, 26, 27, 28, 29, 30, 31,32, or week 33 or more of gestation. In other embodiments, the subject sample may be obtained late in gestation, for example, at or after 34 weeks of gestation, e.g. at week 35, 36, 37, 38, 39, 40, or week 41 of gestation.

Once a sample is obtained, it can be used directly, frozen, or maintained in appropriate culture medium for short periods of time. Typically the samples will be from human patients, although animal models may find use, e.g. equine, bovine, porcine, canine, feline, rodent, e.g. mice, rats, hamster, primate, etc. Any convenient tissue sample that demonstrates the differential representation in a patient with preeclampsia of the one or more preeclampsia markers disclosed herein may be evaluated in the subject methods. Typically, a suitable sample source will be derived from fluids into which the molecular entity of interest, i.e. the RNA transcript or protein, has been released.

The subject sample may be treated in a variety of ways so as to enhance detection of the one or more preeclampsia markers. For example, where the sample is blood, the red blood cells may be removed from the sample (e.g., by centrifugation) prior to assaying. Such a treatment may serve to reduce the non-specific background levels of detecting the level of a preeclampsia marker using an affinity reagent. Detection of a preeclampsia marker may also be enhanced by concentrating the sample using procedures well known in the art (e.g. acid precipitation, alcohol precipitation, salt precipitation, hydrophobic precipitation, filtration (using a filter which is capable of retaining molecules greater than 30 kD, e.g. Centrim 30™), affinity purification). In some embodiments, the pH of the test and control samples will be adjusted to, and maintained at, a pH which approximates neutrality (i.e. pH 6.5-8.0). Such a pH adjustment will prevent complex formation, thereby providing a more accurate quantitation of the level of marker in the sample. In embodiments where the sample is urine, the pH of the sample is adjusted and the sample is concentrated in order to enhance the detection of the marker.

In practicing the subject methods, the level(s) of preeclampsia marker(s) in the biological sample from an individual are evaluated. The level of one or more preeclampsia markers in the subject sample may be evaluated by any convenient method. For example, preeclampsia gene expression levels may be detected by measuring the levels/amounts of one or more nucleic acid transcripts, e.g. mRNAs, of one or more preeclampsia genes. Protein markers may be detected by measuring the levels/amounts of one or more proteins/polypeptides. The terms “evaluating”, “assaying”, “measuring”, “assessing,” and “determining” are used interchangeably to refer to any form of measurement, including determining if an element is present or not, and including both quantitative and qualitative determinations. Evaluating may be relative or absolute.

For example, the level of at least one preeclampsia marker may be evaluated by detecting in a sample the amount or level of one or more proteins/polypeptides or fragments thereof to arrive at a protein level representation. The terms “protein” and “polypeptide” as used in this application are interchangeable. “Polypeptide” refers to a polymer of amino acids (amino acid sequence) and does not refer to a specific length of the molecule. Thus peptides and oligopeptides are included within the definition of polypeptide. This term also refers to or includes post-translationally modified polypeptides, for example, glycosylated polypeptide, acetylated polypeptide, phosphorylated polypeptide and the like. Included within the definition are, for example, polypeptides containing one or more analogs of an amino acid, polypeptides with substituted linkages, as well as other modifications known in the art, both naturally occurring and non-naturally occurring.

When protein levels are to be detected, any convenient protocol for evaluating protein levels may be employed wherein the level of one or more proteins in the assayed sample is determined. For example, one representative and convenient type of protocol for assaying protein levels is ELISA. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tweenor PBSATriton-X 100, which also tend to assist in the reduction of nonspecific background, and allowing the sample to incubate for about 2-4 hrs at temperatures on the order of about 25°−27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hrs at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatograpic column or filter with a wash solution or solvent.

Alternatively, non-ELISA based-methods for measuring the levels of one or more proteins in a sample may be employed. Representative examples include but are not limited to mass spectrometry, proteomic arrays, xMAP™ microsphere technology, flow cytometry, western blotting, and immunohistochemistry.

As another example, the level of at least one preeclampsia marker may be evaluated by detecting in a patient sample the amount or level of one or more RNA transcripts or a fragment thereof encoded by the gene of interest to arrive at a nucleic acid marker representation. The level of nucleic acids in the sample may be detected using any convenient protocol. While a variety of different manners of detecting nucleic acids are known, such as those employed in the field of differential gene expression analysis, one representative and convenient type of protocol for generating marker representations is array-based gene expression profiling protocols. Such applications are hybridization assays in which a nucleic acid that displays “probe” nucleic acids for each of the genes to be assayed/profiled in the marker representation to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the marker representations employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., marker representation (e.g., in the form of a transcriptosome), may be both qualitative and quantitative.

Alternatively, non-array based methods for quantitating the level of one or more nucleic acids in a sample may be employed, including those based on amplification protocols, e.g., Polymerase Chain Reaction (PCR)-based assays, including quantitative PCR, reverse-transcription PCR (RT-PCR), real-time PCR, and the like.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

The resultant data provides information regarding levels in the sample for each of the markers that have been probed, wherein the information is in terms of whether or not the marker is present and, typically, at what level, and wherein the data may be both qualitative and quantitative. As such, where detection is qualitative, the methods provide a reading or evaluation, e.g., assessment, of whether or not the target marker, e.g., nucleic acid or protein, is present in the sample being assayed. In yet other embodiments, the methods provide a quantitative detection of whether the target marker is present in the sample being assayed, i.e., an evaluation or assessment of the actual amount or relative abundance of the target analyte, e.g., nucleic acid or protein in the sample being assayed. In such embodiments, the quantitative detection may be absolute or, if the method is a method of detecting two or more different analytes, e.g., target nucleic acids or protein, in a sample, relative. As such, the term “quantifying” when used in the context of quantifying a target analyte, e.g., nucleic acid(s) or protein(s), in a sample can refer to absolute or to relative quantification. Absolute quantification may be accomplished by inclusion of known concentration(s) of one or more control analytes and referencing the detected level of the target analyte with the known control analytes (e.g., through generation of a standard curve). Alternatively, relative quantification can be accomplished by comparison of detected levels or amounts between two or more different target analytes to provide a relative quantification of each of the two or more different analytes, e.g., relative to each other.

Once the level of the one or more preeclampsia markers has been determined, the measurement(s) may be analyzed in any of a number of ways to obtain a preeclampsia marker level representation.

For example, the measurements of the one or more preeclampsia markers may be analyzed individually to develop a preeclampsia profile. As used herein, a “preeclampsia profile” is the normalized level of one or more preeclampsia markers in a patient sample, for example, the normalized level of serological protein concentrations in a patient sample. A profile may be generated by any of a number of methods known in the art. For example, the level of each marker may be log₂ transformed and normalized relative to the expression of a selected housekeeping gene, or relative to the signal across a whole panel, etc. Other methods of calculating a preeclampsia profile will be readily known to the ordinarily skilled artisan.

As another example, the measurements of a panel of preeclampsia markers may be analyzed collectively to arrive at a single preeclampsia score. By a “preeclampsia score” it is meant a single metric value that represents the weighted levels of each of the preeclampsia markers in the preeclampsia panel. As such, in some embodiments, the subject method comprises detecting the level of markers of a preeclampsia panel in the sample, and calculating a preeclampsia score based on the weighted levels of the preeclampsia markers. A preeclampsia score for a patient sample may be calculated by any of a number of methods and algorithms known in the art for calculating biomarker scores. For example, weighted marker levels, e.g. log₂ transformed and normalized marker levels that have been weighted by, e.g., multiplying each normalized marker level to a weighting factor, may be totaled and in some cases averaged to arrive at a single value representative of the panel of preeclampsia markers analyzed.

In some instances, the weighting factor, or simply “weight” for each marker in a panel may be a reflection of the change in analyte level in the sample. For example, the analyte level of each preeclampsia marker may be log transformed and weighted either as 1 (for those markers that are increased in level in preeclampsia) or −1 (for those markers that are decreased in level in preeclampsia), and the ratio between the sum of increased markers as compared to decreased markers determined to arrive at a preeclampsia signature. In other instances, the weights may be reflective of the importance of each marker to the specificity, sensitivity and/or accuracy of the marker panel in making the diagnostic, prognostic, or monitoring assessment. Such weights may be determined by any convenient statistical machine learning methodology, e.g. Principle Component Analysis (PCA), linear regression, support vector machines (SVMs), and/or random forests of the dataset from which the sample was obtained may be used. In some instances, weights for each marker are defined by the dataset from which the patient sample was obtained. In other instances, weights for each marker may be defined based on a reference dataset, or “training dataset”.

For example, as disclosed in the examples here, in a preeclampsia panel comprising the markers Activin A, ENG, EPCR, and PIGF, ENG and PIGF levels are most significant, level of Activin A is moderately important, and level of EPCR is less significant. As such, one example of an algorithm that may be used to arrive at a preeclampsia score would be an algorithm that considers ENG and PIGF levels most strongly; that considers Activin A level more modestly; that considers EPCR least.

These methods of analysis may be readily performed by one of ordinary skill in the art by employing a computer-based system, e.g. using any hardware, software and data storage medium as is known in the art, and employing any algorithms convenient for such analysis. For example, data mining algorithms can be applied through “cloud computing”, smartphone based or client-server based platforms, and the like.

In certain embodiments the expression, e.g. polypeptide level, of only one marker is evaluated to produce a marker level representation. In yet other embodiments, the levels of two or more, i.e. a panel, markers, e.g., 3 or more, 4 or more, 5 or more markers is evaluated. Accordingly, in the subject methods, the expression of at least one marker in a sample is evaluated. In certain embodiments, the evaluation that is made may be viewed as an evaluation of the proteome, as that term is employed in the art.

In some instances, the subject methods of determining or obtaining a preeclampsia marker representation (e.g. preeclampsia profile or preeclampsia score) for a subject further comprise providing the preeclampsia marker representation as a report. Thus, in some instances, the subject methods may further include a step of generating or outputting a report providing the results of a preeclampsia marker evaluation in the sample, which report can be provided in the form of an electronic medium (e.g., an electronic display on a computer monitor), or in the form of a tangible medium (e.g., a report printed on paper or other tangible medium). Any form of report may be provided, e.g. as known in the art or as described in greater detail below.

### Utility

Preeclampsia marker level representations so obtained find many uses. For example, the marker level representation may be employed to diagnose a preeclampsia; that is, to provide a determination as to whether a subject is affected by preeclampsia, the type of preeclampsia, the severity of preeclampsia, etc. In some instances, the subject may present with clinical symptoms of preeclampsia, e.g. elevated blood pressure (e.g. 140/90 mm/Hg or higher), proteinuria, sudden weight gain (over 1-2 days or more than 2 pounds a week), water retention (edema), elevated liver enzymes, and/or thrombocytopenia (a depressed platelet count less than 100,000). In other instances, subject may be asymptomatic for preeclampsia but has risk factors associated with preeclampsia, e.g. a medical condition such as gestational diabetes, type I diabetes, obesity, chronic hypertension, renal disease, a thrombophilia; African-American or Filipino descent; age of greater than 35 years or less than 20 years; a family history of preeclampsia; nulliparity; preeclampsia in a previous pregnancy; and/or stress. In yet other instances, the subject may be asymptomatic for preeclampsia and have no risk factors associated with preeclampsia.

As another example, the preeclampsia marker level representation may be employed to prognose a preeclampsia; that is, to provide a preeclampsia prognosis. For example, the preeclampsia marker level representation may be used to predict a subject's susceptibility, or risk, of developing preeclampsia. By “predicting if the individual will develop preeclampsia”, it is meant determining the likelihood that an individual will develop preeclampsia in the next week, in the next 2 weeks, in the next 3 weeks, in the next 5 weeks, in the next 2 months, in the next 3 months, e.g. during the remainder of the pregnancy. The preeclampsia marker level representation may be used to predict the course of disease progression and/or disease outcome, e.g. expected onset of the preeclampsia, expected duration of the preeclampsia, expectations as to whether the preeclampsia will develop into eclampsia, etc. The preeclampsia marker level representation may be used to predict a subject's responsiveness to treatment for the preeclampsia, e.g., positive response, a negative response, no response at all.

As another example, the preeclampsia marker level representation may be employed to monitor a preeclampsia. By “monitoring” a preeclampsia, it is generally meant monitoring a subject's condition, e.g. to inform a preeclampsia diagnosis, to inform a preeclampsia prognosis, to provide information as to the effect or efficacy of a preeclampsia treatment, and the like.

As another example, the preeclampsia marker level representation may be employed to determine a treatment for a subject. The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. The subject therapy may be administered prior to the symptomatic stage of the disease, and in some cases after the symptomatic stage of the disease. The terms “individual,” “subject,” “host,” and “patient,” are used interchangeably herein and refer to any mammalian subject for whom diagnosis, treatment, or therapy is desired, particularly humans. Preeclampsia treatments are well known in the art, and may include bed rest, drinking extra water, a low salt diet, medicine to control blood pressure, corticosteroids, inducing pregnancy, and the like.

In some embodiments, the subject methods of providing a preeclampsia assessment, e.g. diagnosing a preeclampsia, prognosing a preeclampsia, monitoring the preeclampsia, treating the preeclampsia, and the like, may comprise comparing the obtained preeclampsia marker level representation to a preeclampsia phenotype determination element to identify similarities or differences with the phenotype determination element, where the similarities or differences that are identified are then employed to provide the preeclampsia assessment, e.g. diagnose the preeclampsia, prognose the preeclampsia, monitor the preeclampsia, determine a preeclampsia treatment, etc. By a “phenotype determination element” it is meant an element, e.g. a tissue sample, a marker profile, a value (e.g. score), a range of values, and the like that is representative of a phenotype (in this instance, a preeclampsia phenotype) and may be used to determine the phenotype of the subject, e.g. if the subject is healthy or is affected by preeclampsia, if the subject has a preeclampsia that is likely to progress to eclampsia, if the subject has a preeclampsia that is responsive to therapy, etc.

For example, a preeclampsia phenotype determination element may be a sample from an individual that has or does not have preeclampsia, which may be used, for example, as a reference/control in the experimental determination of the marker level representation for a given subject. As another example, a preeclampsia phenotype determination element may be a marker level representation, e.g. marker profile or score, which is representative of a preeclampsia state and may be used as a reference/control to interpret the marker level representation of a given subject. The phenotype determination element may be a positive reference/control, e.g., a sample or marker level representation thereof from a pregnant woman that has preeclampsia, or that will develop preeclampsia, or that has preeclampsia that is manageable by known treatments, or that has preeclampsia that has been determined to be responsive only to the delivery of the baby. Alternatively, the phenotype determination element may be a negative reference/control, e.g. a sample or marker level representation thereof from a pregnant woman that has not developed preeclampsia, or an woman that is not pregnant. Phenotype determination elements are preferably the same type of sample or, if marker level representations, are obtained from the same type of sample as the sample that was employed to generate the marker level representation for the individual being monitored. For example, if the serum of an individual is being evaluated, the phenotype determination element would preferably be of serum.

In certain embodiments, the obtained marker level representation is compared to a single phenotype determination element to obtain information regarding the individual being tested for preeclampsia. In other embodiments, the obtained marker level representation is compared to two or more phenotype determination elements. For example, the obtained marker level representation may be compared to a negative reference and a positive reference to obtain confirmed information regarding if the individual will develop preeclampsia. As another example, the obtained marker level representation may be compared to a reference that is representative of a preeclampsia that is responsive to treatment and a reference that is representative of a preeclampsia that is not responsive to treatment to obtain information as to whether or not the patient will be responsive to treatment.

The comparison of the obtained marker level representation to the one or more phenotype determination elements may be performed using any convenient methodology, where a variety of methodologies are known to those of skill in the art. For example, those of skill in the art of ELISAs will know that ELISA data may be compared by, e.g. normalizing to standard curves, comparing normalized values, etc. The comparison step results in information regarding how similar or dissimilar the obtained marker level profile is to the control/reference profile(s), which similarity/dissimilarity information is employed to, for example, predict the onset of a preeclampsia, diagnose preeclampsia, monitor a preeclampsia patient, etc. Similarly, those of skill in the art of arrays will know that array profiles may be compared by, e.g., comparing digital images of the expression profiles, by comparing databases of expression data, etc. patents describing ways of comparing expression profiles include, but are not limited to, U.S. Pat. Nos. 6,308,170 and 6,228,575, the disclosures of which are herein incorporated by reference. Methods of comparing marker level profiles are also described above. Similarity may be based on relative marker levels, absolute marker levels or a combination of both. In certain embodiments, a similarity determination is made using a computer having a program stored thereon that is designed to receive input for a marker level representation obtained from a subject, e.g., from a user, determine similarity to one or more reference profiles or reference scores, and return an preeclampsia prognosis, e.g., to a user (e.g., lab technician, physician, pregnant individual, etc.). Further descriptions of computer-implemented aspects of the invention are described below. In certain embodiments, a similarity determination may be based on a visual comparison of the marker level representation, e.g. preeclampsia score, to a range of phenotype determination elements, e.g. a range of preeclampsia scores, to determine the reference preeclampsia score that is most similar to that of the subject. Depending on the type and nature of the phenotype determination element to which the obtained marker level profile is compared, the above comparison step yields a variety of different types of information regarding the cell/bodily fluid that is assayed. As such, the above comparison step can yield a positive/negative prediction of the onset of preeclampsia, a positive/negative diagnosis of preeclampsia, a characterization of a preeclampsia, information on the responsiveness of a preeclampsia to treatment, and the like.

In other embodiments, the marker level representation is employed directly, i.e. without comparison to a phenotype determination element, to make a preeclampsia prognosis, preeclampsia diagnosis, or monitor a preeclampsia. For example, a patient may be predicted to develop preeclampsia if the concentration of Activin A in the patient's serum is about 5.5 ng/ml or greater; if the concentration of ENG in the patient's serum is about 17 ng/ml or greater.

In some embodiments, the subject methods of providing a preeclampsia assessment, e.g. diagnosing a preeclampsia, prognosing a preeclampsia, monitoring the preeclampsia, and the like, may comprise additional assessment(s) that are employed in conjunction with the subject marker level representation. For example, the subject methods may further comprise measuring one or more clinical parameters/factors associated with preeclampsia, e.g. blood pressure, urine protein, weight changes, water retention (edema), liver enzyme levels, and platelet count. For example, a subject maybe assessed for one or more clinical symptoms, e.g. hypertension, proteinuria, etc., at about week 14 or more of gestation, e.g. week 15, 16,17,18,19, 20, 21,22, 23, 24, 25, 26, 27, 28, 29, 30, 31,32, 33, 34 35, 36, 37, 38, 39, 40 or more of gestation, wherein a positive outcome of the clinical assessment (i.e. the detection of one or more symptoms associated with preeclampsia) is used in combination with the marker level representation to provide a preeclampsia diagnosis, a preeclampsia prognosis, to monitor the preeclampsia, etc. In some instances, the clinical parameters may be measured prior to obtaining the preeclampsia marker level representation, for example, to inform the artisan as to whether a preeclampsia marker level representation should be obtained, e.g. to make or confirm a preeclampsia diagnosis. In some instances, the clinical parameters may be measured after obtaining the preeclampsia marker level representation, e.g. to monitor a preeclampsia.

As another example, the subject methods of providing a preeclampsia assessment may further comprise assessing one or more factors associated with the risk of developing preeclampsia. Non-limiting examples of preeclampsia risk factors include, for example, a medical condition such as gestational diabetes, obesity, chronic hypertension, renal disease, a thrombophilia; age of greater than 35 years or less than 20 years; a family history of preeclampsia; nulliparity; preeclampsia in a previous pregnancy; and stress. For example, a subject maybe assessed for one or more risk factors, e.g. medical condition, family history, etc., when pregnancy is first confirmed or thereafter, wherein a positive outcome of the risk assessment (i.e. the determination of one or more risk factors associated with preeclampsia) is used in combination with the marker level representation to provide a preeclampsia diagnosis, a preeclampsia prognosis, to monitor the preeclampsia, etc.

The subject methods may be employed for a variety of different types of subjects. In many embodiments, the subjects are within the class mammalian, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the animals or hosts, i.e., subjects (also referred to herein as patients), are humans.

In some embodiments, the subject methods of providing a preeclampsia assessment include providing a diagnosis, prognosis, or result of the monitoring. In some embodiments, the preeclampsia assessment of the present disclosure is provided by providing, i.e. generating, a written report that includes the artisan's assessment, for example, the artisan's determination of whether the patient is currently affected by preeclampsia, of the type, stage, or severity of the subject's preeclampsia, etc. (a “preeclampsia diagnosis”); the artisan's prediction of the patient's susceptibility to developing preeclampsia, of the course of disease progression, of the patient's responsiveness to treatment, etc. (i.e., the artisan's “preeclampsia prognosis”); or the results of the artisan's monitoring of the preeclampsia. Thus, the subject methods may further include a step of generating or outputting a report providing the results of an artisan's assessment, which report can be provided in the form of an electronic medium (e.g., an electronic display on a computer monitor), or in the form of a tangible medium (e.g., a report printed on paper or other tangible medium). Any form of report may be provided, e.g. as known in the art or as described in greater detail below.

### Reports

A “report,” as described herein, is an electronic or tangible document which includes report elements that provide information of interest relating to the assessment of a subject and its results. In some embodiments, a subject report includes at least a preeclampsia marker representation, e.g. a preeclampsia profile or a preeclampsia score, as discussed in greater detail above. In some embodiments, a subject report includes at least an artisan's preeclampsia assessment, e.g. preeclampsia diagnosis, preeclampsia prognosis, an analysis of a preeclampsia monitoring, a treatment recommendation, etc. A subject report can be completely or partially electronically generated. A subject report can further include one or more of: 1) information regarding the testing facility; 2) service provider information; 3) patient data; 4) sample data; 5) an assessment report, which can include various information including: a) reference values employed, and b) test data, where test data can include, e.g., a protein level determination; 6) other features.

The report may include information about the testing facility, which information is relevant to the hospital, clinic, or laboratory in which sample gathering and/or data generation was conducted. Sample gathering can include obtaining a fluid sample, e.g. blood, saliva, urine etc.; a tissue sample, e.g. a tissue biopsy, etc. from a subject. Data generation can include measuring the marker concentration in preeclampsia patients versus healthy individuals, i.e. individuals that do not have and/or do not develop preeclampsia. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a patient data section, including patient medical history (which can include, e.g., age, race, serotype, prior preeclampsia episodes, and any other characteristics of the pregnancy), as well as administrative patient data such as information to identify the patient (e.g., name, patient date of birth (DOB), gender, mailing and/or residence address, medical record number (MRN), room and/or bed number in a healthcare facility), insurance information, and the like), the name of the patient's physician or other health professional who ordered the monitoring assessment and, if different from the ordering physician, the name of a staff physician who is responsible for the patient's care (e.g., primary care physician).

The report may include a sample data section, which may provide information about the biological sample analyzed in the monitoring assessment, such as the source of biological sample obtained from the patient (e.g. blood, saliva, or type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu). The report may include a results section. For example, the report may include a section reporting the results of a protein level determination assay (e.g., “5.0 ng/ml Activin A in serum”), or a calculated preeclampsia score.

The report may include an assessment report section, which may include information generated after processing of the data as described herein. The interpretive report can include a prediction of the likelihood that the subject will develop preeclampsia. The interpretive report can include a diagnosis of preeclampsia. The interpretive report can include a characterization of preeclampsia. The assessment portion of the report can optionally also include a recommendation(s). For example, where the results indicate that preeclampsia is likely, the recommendation can include a recommendation that diet be altered, blood pressure medicines administered, etc., as recommended in the art.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g. a calculated preeclampsia marker level representation; a prediction, diagnosis or characterization of preeclampsia).

### Reagents, Systems and Kits

Also provided are reagents, systems and kits thereof for practicing one or more of the above-described methods. The subject reagents, systems and kits thereof may vary greatly. Reagents of interest include reagents specifically designed for use in producing the above-described marker level representations of preeclampsia markers from a sample, for example, one or more detection elements, e.g. antibodies or peptides for the detection of protein, oligonucleotides for the detection of nucleic acids, etc. In some instances, the detection element comprises a reagent to detect the expression of a single preeclampsia marker, for example, the detection element may be a dipstick, a plate, an array, or cocktail that comprises one or more detection elements, e.g. one or more antibodies, one or more oligonucleotides, one or more sets of PCR primers, etc. which may be used to detect the expression of one or more preeclampsia marker simultaneously,

One type of reagent that is specifically tailored for generating marker level representations, e.g. preeclampsia marker level representations, is a collection of antibodies that bind specifically to the protein markers, e.g. in an ELISA format, in an xMAP™ microsphere format, on a proteomic array, in suspension for analysis by flow cytometry, by western blotting, by dot blotting, or by immunohistochemistry. Methods for using the same are well understood in the art. These antibodies can be provided in solution. Alternatively, they may be provided pre-bound to a solid matrix, for example, the wells of a multi-well dish or the surfaces of xMAP microspheres.

Another type of such reagent is an array of probe nucleic acids in which the genes of interest are represented. A variety of different array formats are known in the art, with a wide variety of different probe structures, substrate compositions and attachment technologies (e.g., dot blot arrays, microarrays, etc.). Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280.

Another type of reagent that is specifically tailored for generating marker level representations of genes, e.g. preeclampsia genes, is a collection of gene specific primers that is designed to selectively amplify such genes (e.g., using a PCR-based technique, e.g., real-time RT-PCR). Gene specific primers and methods for using the same are described in U.S. Pat. No. 5,994,076, the disclosure of which is herein incorporated by reference.

Of particular interest are arrays of probes, collections of primers, or collections of antibodies that include probes, primers or antibodies (also called reagents) that are specific for at least 1 gene/protein selected from the group consisting of Activin A, ENG, EPCR, PIGF, and sFlt-1, or a biochemical substrate specific for the cofactor/prosthetic group heme, in some instances for a plurality of these genes/polypeptides, e.g., at least 2, 3, 4, 5, 6, 7, 8 or more genes/polypeptides. In certain embodiments, the collection of probes, primers, or antibodies includes reagents specific for Activin A, ENG, EPCR, PIGF, and sFlt-1 as well as a biochemical substrate specific for heme. The subject probe, primer, or antibody collections or reagents may include reagents that are specific only for the genes/proteins/cofactors that are listed above, or they may include reagents specific for additional genes/proteins/cofactors that are not listed above, such as probes, primers, or antibodies specific for genes/proteins/cofactors whose expression pattern are known in the art to be associated with preeclampsia, e.g. and sFlt-1 (VEGF-RI) and PIGF.

In some instances, a system may be provided. As used herein, the term “system” refers to a collection of reagents, however compiled, e.g., by purchasing the collection of reagents from the same or different sources. In some instances, a kit may be provided. As used herein, the term “kit” refers to a collection of reagents provided, e.g., sold, together. For example, the nucleic acid- or antibody-based detection of the sample nucleic acid or protein, respectively, may be coupled with an electrochemical biosensor platform that will allow multiplex determination of these biomarkers for personalized preeclampsia care.

The systems and kits of the subject invention may include the above-described arrays, gene-specific primer collections, or protein-specific antibody collections. The systems and kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. labeled secondary antibodies, streptavidin-alkaline phosphatase conjugate, chemifluorescent or chemiluminescent substrate, and the like.

The subject systems and kits may also include one or more preeclampsia phenotype determination elements, which element is, in many embodiments, a reference or control sample or marker representation that can be employed, e.g., by a suitable experimental or computing means, to make a preeclampsia prognosis based on an “input” marker level profile, e.g., that has been determined with the above described marker determination element. Representative preeclampsia phenotype determination elements include samples from an individual known to have or not have preeclampsia, databases of marker level representations, e.g., reference or control profiles or scores, and the like, as described above.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

As the leading cause of maternal morbidity and mortality, preeclampsia (PE) is a pregnancy-related vascular disorder affecting 5%-8% of all pregnancies (Berg et al. Overview of maternal morbidity during hospitalization for labor and delivery in the United States: 1993-1997 and 2001-2005. Obstetrics and gynecology 2009; 113:1075-81; Mackay et al. Pregnancy-related mortality from preeclampsia and eclampsia. Obstetrics and gynecology 2001; 97:533-8). PE, which often causes fetal growth restriction and pre-term delivery as well as fetal mortality and morbidity, can be remedied by delivery of the placenta and fetus (Powe et al. Preeclampsia, a disease of the maternal endothelium: the role of antiangiogenic factors and implications for later cardiovascular disease. Circulation 2011; 123:2856-69). The etiology of PE is incompletely understood. Current diagnosis of PE is based on the signs of hypertension and proteinuria (Gynecologists ACOOA ACOG practice bulletin. Diagnosis and management of preeclampsia and eclampsia. Number 33, January 2002. Obstetrics and gynecology 2002; 99:159-67), but lacks sensitivity and specificity and carries a poor prognosis for adverse maternal and fetal outcomes (Zhang et al. Prediction of adverse outcomes by common definitions of hypertension in pregnancy. Obstetrics and gynecology 2001; 97:261-7). Thus, there is a need to identify PE biomarkers that can provide a definitive diagnosis with the opportunity for better monitoring of the condition's progression, and thus improved outcomes and economic benefits.

Although the pathophysiology remains largely elusive, PE is a multisystem disorder of pregnancy with the placenta playing a pivotal role. Investigators have used genetic, genomic and proteomic approaches to compare PE and control placental tissues. Transcriptional profiling of case-control samples has identified disease-specific expression patterns, canonical pathways and gene-gene networks (Lapaire et al. Microarray screening for novel preeclampsia biomarker candidates. Fetal diagnosis and therapy 2012; 31:147-53; Nishizawa et al. Microarray analysis of differentially expressed fetal genes in placenta tissue derived from early and late onset severe preeclampsia. Placenta 2007; 28:487-97; Loset et al. transcriptional profile of the decidua in preeclampsia. American journal of obstetrics and gynecology 2011; 204:84 e1-27; Johansson et al. Partial correlation network analyses to detect altered gene interactions in human disease: using preeclampsia as a model. Human genetics 2011; 129:25-34; Sitras et al. Differential placental gene expression in severe preeclampsia. Placenta 2009; 30:424-33; Tsai et al. Transcriptional profiling of human placentas from pregnancies complicated by preeclampsia reveals disregulation of sialic acid acetylesterase and immune signaling pathways. Placenta 2011; 32:175-82; Winn et al. Severe preeclampsia-related changes in gene expression at the maternal-fetal interface include sialic acid-binding immunoglobulin-like lectin-6 and pappalysin-2. Endocrinology 2009; 150:452-62). Proteomics-based biomarker studies (Kolia et al. Quantitative proteomic (iTRAQ) analysis of 1st trimester maternal plasma samples in pregnancies at risk for preeclampsia. Journal of biomedicine & biotechnology 2012; 2012:305964; Mary et al. Dynamic proteome in enigmatic preeclampsia: an account of molecular mechanisms and biomarker discovery. Proteomics Clinical applications 2012; 6:79-90; Carty et al. Urinary proteomics for prediction of preeclampsia. Hypertension 2011; 57:561-9) have also revealed candidate biomarkers for future testing. Placental angiogenic and anti-angiogenic factor imbalance, elevated soluble fms-like tyrosine kinase (sFlt-1) and decreased placental growth factor (PIGF) levels, are suggested in the pathogenesis of PE (Shibata et al. Soluble fms-like tyrosine kinase 1 is increased in preeclampsia but not in normotensive pregnancies with small-for-gestational-age neonates: relationship to circulating placental growth factor. The Journal of clinical endocrinology and metabolism 2005; 90:4895-903; Maynard et al. Excess placental soluble fms-like tyrosine kinase 1 (sFlt-1) may contribute to endothelial dysfunction, hypertension, and proteinuria in preeclampsia. The Journal of clinical investigation 2003; 111:649-58; Wolf et al. Circulating levels of the antiangiogenic marker sFLT-1 are increased in first versus second pregnancies. American journal of obstetrics and gynecology 2005; 193:16-22; Rajakumar et al. Extra-placental expression of vascular endothelial growth factor receptor-1, (Flt-1) and soluble Flt-1 (sFlt-1), by peripheral blood mononuclear cells (PBMCs) in normotensive and preeclamptic pregnant women. Placenta 2005; 26:563-73; Taylor et al. Altered tumor vessel maturation and proliferation in placenta growth factor-producing tumors: potential relationship to post-therapy tumor angiogenesis and recurrence. International journal of cancer Journal international du cancer 2003; 105:158-64; Tidewell et al. Low maternal serum levels of placenta growth factor as an antecedent of clinical preeclampsia. American journal of obstetrics and gynecology 2001; 184:1267-72; Torry et al. Preeclampsia is associated with reduced serum levels of placenta growth factor. American journal of obstetrics and gynecology 1998; 179:1539-44), and the sFlt-1/PIGF ratio has been proposed as a useful index in the diagnosis and management of PE (Stepan et al. [use of angiogenic factors (sflt-1/plgf ratio) to confirm the diagnosis of preeclampsia in clinical routine: First experience]. Zeitschrift fur Geburtshilfe and Neonatologie. 2010; 214:234-238; Verlohren et al. An automated method for the determination of the sflt-1/pigf ratio in the assessment of preeclampsia. Am. J. Obst. And Gyn. 2010; 202:161 e161-161 e111). However, no widely applicable, sensitive and specific molecular PE test in routine clinical practice is currently available.

In light of these considerations, there is a strong rationale and need to discover diagnostic and prognostic biomarkers for PE. We employed a comprehensive unbiased multi-‘omics’ approach, integrating results from microarray multiplex meta-analysis, and proteomic identification by two-dimensional (2D) gel analysis. Our applied parametric method (Morgan et al. Comparison of multiplex meta analysis techniques for understanding the acute rejection of solid organ transplants. BMC bioinformatics 2010; 11 Suppl 9:S6; Chen et al. Differentially expressed RNA from public microarray data identifies serum protein biomarkers for cross-organ transplant rejection and other conditions. PLoS computational biology 2010; 6) in meta-analysis allowed us to identify consistent and significant differential gene expression across experiments to develop biomarkers for downstream experimental validation. Serum proteins are routinely used to diagnose diseases, but sensitive and specific biomarkers are hard to find and may be due to their low serological abundance, which can easily be masked by highly abundant proteins. Our serum protein marker discovery method (Ling et al. Plasma profiles in active systemic juvenile idiopathic arthritis: Biomarkers and biological implications. Proteomics 2010) combines antibody-based serum abundant protein depletion and 2D gel comparative profiling to discover differential protein gel spots between PE and control sera for subsequent protein mass spectrometric identification. We hypothesized that there would be differential serological signatures allowing PE diagnosis. To validate our discovery findings, we tested all the candidates with available ELISA assays, a higher-throughput method. To construct and optimize a sensitive and specific biomarker panel with the least number of protein analytes, a genetic algorithm was used. Close examination of the biomarkers from comparative transcriptomics and proteomics, and their associated pathways led to new hypothesis about their role in PE pathophysiology.

The presented results validated our hypothesis that sensitive and specific serological biomarker panels can be constructed to diagnose PE. To our knowledge, this represents the first study to employ a muti-‘omics’-based biomarker approach to uncover novel PE biomarkers including Activin A, ENG, and EPCR that are comparable to sFlt-1/PIGF ratio in PE discrimination. We believe that the functional significance of these PE biomarkers and their associated pathways will provide new insights into the disease pathogenesis and lead to effective novel therapeutics.

### Materials and Methods

Study Design.

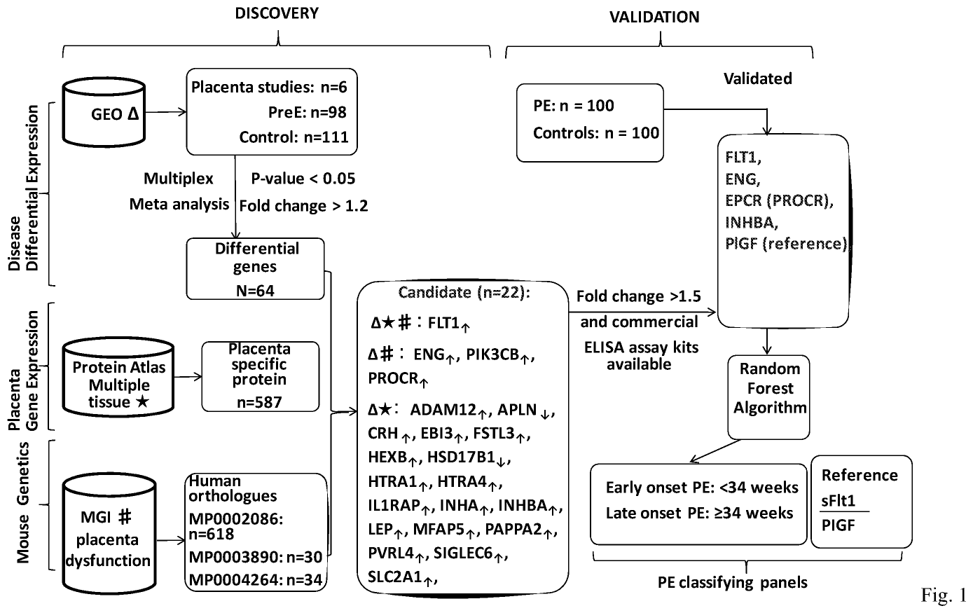

The overall sample allocation, PE biomarker discovery, validation, and predictive panel construction steps are illustrated in FIG. 1. Our study was conducted in two phases: (1) the discovery phase, which included meta analysis of microarray datasets (6 datasets, n=98 PE and n=111 control placenta samples), extraction of placenta specific protein from database of protein atlas and obtainment of human orthologous gene with placenta dysfunction in mouse model from MGI database. (2) the validation phase, which was comprised of the analysis of independent PE (n=100) and control (n=100) cohorts. The candidates for further validation were selected by the fold change >1.5 in the meta analysis and by ELISA assay kits available. The PIGF was selected as positive biomarker.

Clinical cohort design and sample collection. All the serum samples were purchased from R&D systems (MN 55413, https://www.rndsystems.com/), Diagnostica Stago Inc. (NJ 07054, http://www.stago-us.com/). All serum samples were collected after informed consent was obtained, and included detailed case report forms. Patients who were diagnosed with antiphospholipid syndrome (APS), or systemic lupus erythematosus (SLE) or any other concurrent autoimmune diseases or under long term corticosteroid treatment were excluded from PE cohort; patients who were diagnosed with preterm birth, or intrauterine growth retardation (IUGR), HELLP syndrome, and PE were excluded from control cohort. Case (PE) and control (normal pregnant) cohorts were matched for gestational age, ethnicity, and parity.

Multiplex Meta-Analysis of Expression Comparing PE and Control Placentas.

As shown in Table 1 below, six PE placenta expression studies (Nishizawa et al. Microarray analysis of differentially expressed fetal genes in placenta tissue derived from early and late onset severe preeclampsia. Placenta 2007; 28:487-97; Sitras et al. Differential placental gene expression in severe preeclampsia. Placenta 2009; 30:424-33; Tsai et al. Transcriptional profiling of human placentas from pregnancies complicated by preeclampsia reveals disregulation of sialic acid acetylesterase and immune signalling pathways. Placenta 2011; 32:175-82; Nishizawa et al. Comparative gene expression profiling of placentas from patients with severe preeclampsia and unexplained fetal growth restriction. Reproductive biology and endocrinology 2011; 9:107; Blair J D et al. Widespread DNA hypomethylation at gene enhancer regions in placentas associated with early-onset pre-eclampsia. Molecular Human Reproduction 2013; 19:697-708; Jebbink J M et al. Increased glucocerebrosidase expression and activity in preeclamptic placenta. Placenta 2013; 36:160-169) were combined and subjected to multiplex meta-analysis with the method we previously developed (Morgan et al. Comparison of multiplex meta-analysis techniques for understanding the acute rejection of solid organ transplants. BMC bioinformatics 2010; 11 Suppl 9:S6; Chen et al. Differentially expressed RNA from public microarray data identifies serum protein biomarkers for cross-organ transplant rejection and other conditions. PLoS computational biology 2010; 6). For each of the 22,394 genes tested, we calculated the meta-fold change across all studies. Significant genes were selected if they were measured in 5 or more studies and the meta effect p value was less than 0.05 and the meta-fold change higher than 1.2.

Protein Atlas Analysis.

According to Uhlén M et al (Proteomics. Tissue-based map of the human proteome. Science. 2015 Jan. 23; 347(6220):1260419.), the placenta genes were extracted from five tissue categories: tissue enriched, group enriched, tissue enhanced, expressed in all (FPKM>100), mixed. (FPKM>100)

Human Orthologous Gene with Placenta Dysfunction in Mouse Model from MGI Database.

To understand the functional significance of placenta genes in pregnancy disorders, the human orthologous genes whose mouse orthologs were associated with abnormal placental phenotypes when disrupted were obtained from MGI database. Three MGI phenotypes were included: abnormal extraembryonic boundary morphology MP:0003836, abnormal extraembryonic tissue physiology MP:0004264 and abnormal extraembryoic tissue morphology MP:0002086.

ELISA Assays Validating PE Marker Candidates.

All assays were ELISA assays, and performed using commercial kits following vendors' instructions. All assays were performed to measure serum levels of selected analytes: soluble fms-like tyrosine kinase-1(sFlt-1), R&D system Inc. (MN, US); inhibin beta A (Activin A), R&D system Inc. (MN, US); endoglin, CD105 (ENG), R&D system Inc. (MN, US); Endothelial protein C receptor (EPCR), Diagnostica Stago Inc. (NJ, US); placenta Growth factor (PIGF), R&D system Inc. (MN, US).

Statistical Analyses.

Patient demographic and clinical data were analyzed using the “Epidemiological calculator” (R epicalc package). Student's t-test and Mann-Whitney U-test were performed to calculate p values for continuous variables, and Fisher's exact test and Chi-squared test were used for comparative analysis of categorical variables. A group of clinical risk factors of preeclampsia were determined by literature review, and their impacts on preeclampsia diagnosis were explored by uni- and multivariate analysis. Forest plotting with R rmeta package was used both to represent the placental expression meta analysis and to graphically summarize the serum protein ELISA results. Case (PE) and control samples are not paired; thus the initial serum protein forest plot analysis should be interpreted with caution. Bootstrapping method was used to create “paired” samples from case and control groups for the subsequent forest plotting analysis of the ELISA results. Therefore, serum protein forest plot analysis provides an overall effect estimation of each analyte's capability in discriminating PE and normal pregnant control subjects. Hypothesis testing was performed using Student's t-test (two tailed) and Mann-Whitney U-test (two tailed), and local FDR (Efron et al. Empirical bayes analysis of microarray experiment. J Am Stat Assoc 2001; 96:1151-60) to correct for multiple hypothesis testing issues. Biomarker feature selection and panel optimization was performed using a genetic algorithm (R genalg package). The predictive performance of each biomarker panel analysis was evaluated by ROC curve analysis (Zweig et al. Receiver-operating characteristic (ROC) plots: a fundamental evaluation tool in clinical medicine. Clinical chemistry 1993; 39:561-77; Sing et al. ROCR: visualizing classifier performance in R. Bioinformatics 2005; 21:3940-1). The biomarker panel score was defined as the natural logarithm of the ratio between the geometric means of the respective up- and down-regulated protein biomarkers in the maternal circulation. A composite panel combining all significant biomarkers was developed using random forest algorithm, and evaluated by ROC AUC performance.

### Results

Multi-‘Omics’-Based Discovery Revealing PE Marker Candidates.

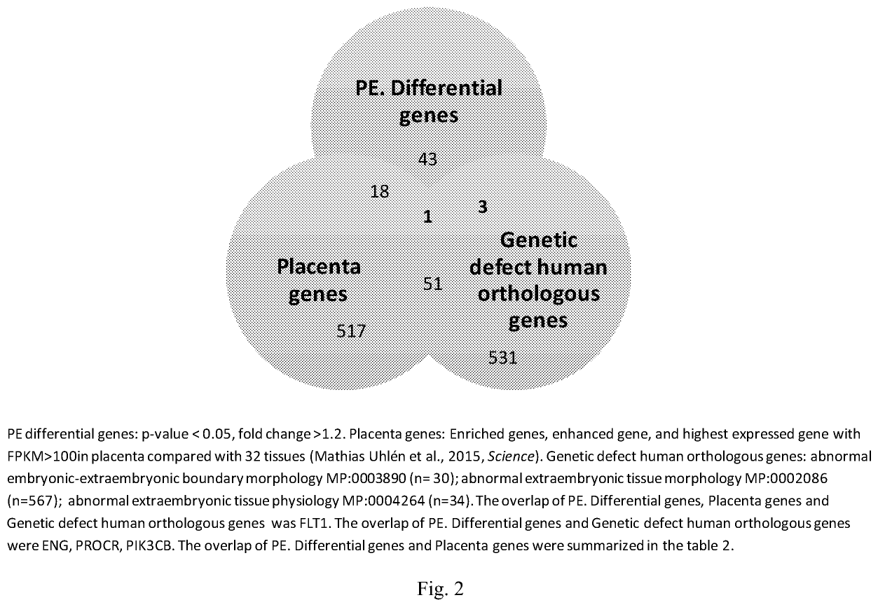

As shown in FIGS. 1 and 2 and Table 2, previous placental expression studies were combined for a multiplex meta-analysis, as well as Protein Atlas analysis and human orthologous gene analysis, to discover biomarker candidates diagnosing PE from normal controls. This effort identified Activin A, ENG, PROCR (EPCR), and sFlt-1 as differential placental biomarkers for PE. PIGF was used as a reference biomarker.

Sample Characteristics.

The PE and control subjects used for serological protein biomarker validation can be divided into early (PE, n=60; control, n=60 with gestation weeks early than 34) and late (PE, n=40; control, n=40 with gestation weeks at or later than 34) gestation groups. As summarized in Table 3 and Table 4 below, no significant differences (p value<0.05) in age (p value, early 0.38, late 0.81, overall 0.59), gestational age (p value, early 0.99, late 0.99, overall 0.99) at enrollment, ethnicity (p value, early 0.99, overall 0.99), or subjects' concurrent medical conditions and other clinical features (p value, overall 0.061) were observed. The gap between the blood sample collection and the delivery was 2.7±3.7 weeks for PE and 8.1±6.1 weeks for control subjects at early stage, and 0.6±0.8 weeks for PE and 2.1±2.5 weeks for control subjects at late stage.

The PE patients were diagnosed with preeclampsia characterized by both hypertension and proteinuria. As shown in Table 5, 100% and 91% of the 100 PE patients had hypertension and proteinuria, respectively; 12% of them had headache; 59% of them had edema; and 4% of them had other additional symptoms. Other characteristics, including body mass index (BMI, prior to pregnancy), blood pressure (BP), protein/creatinine ratio (PCR), pregnancy history, proteinuria, maternal height and weight, and delivery outcomes were shown in Table 6.

Analysis on Risk Factors of Preeclampsia.

A group of risk factors including BMI, age, and concurrent diabetes during pregnancy were selected by literature review. The impact of these risk factors was investigated by univariate and multivariate analysis at early, late, and overall stages, with or without adjustment of gestational age at blood sample collection, respectively (Tables 7-10). Results of odds ratio and hazard ratio showed that BMI had significant, positive impact (p<0.05) on preeclampsia.

Biomarker Validation Using PE and Control Maternal Serum Samples.

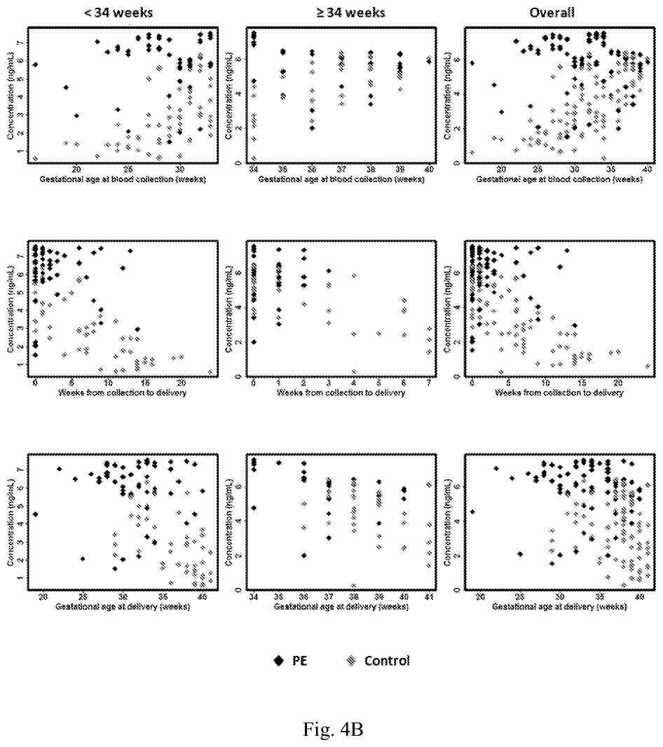

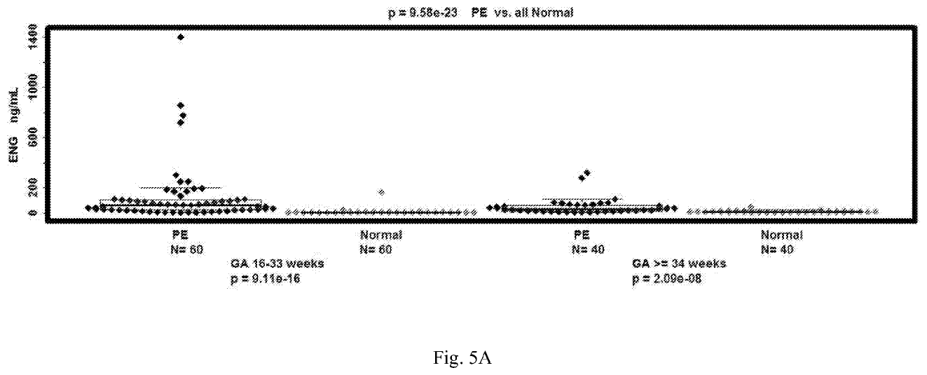

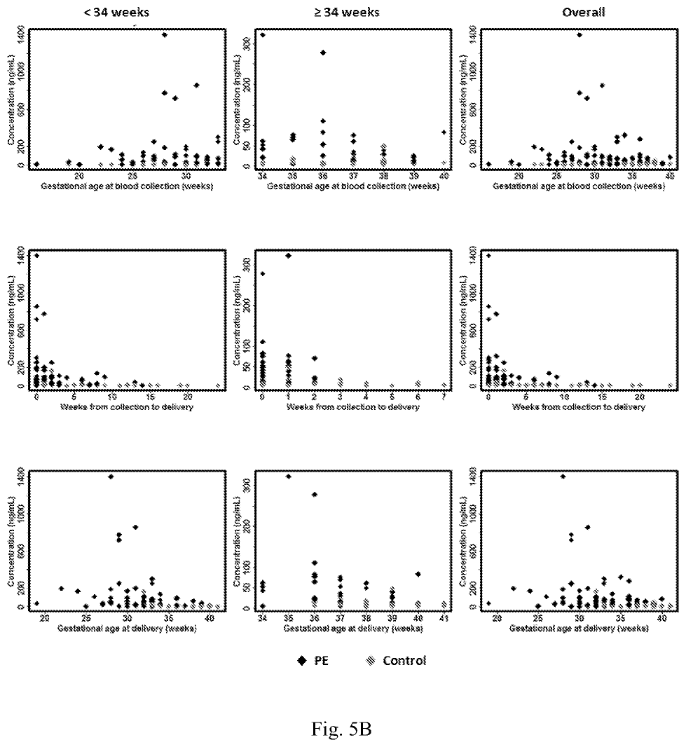

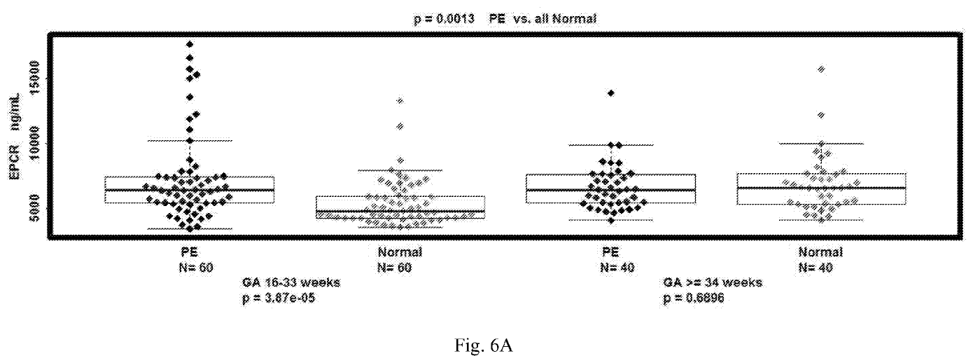

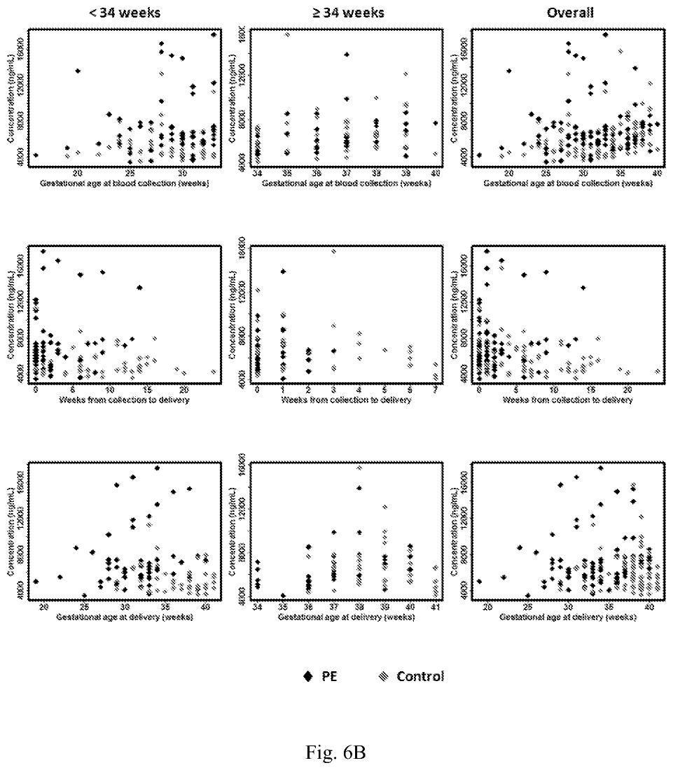

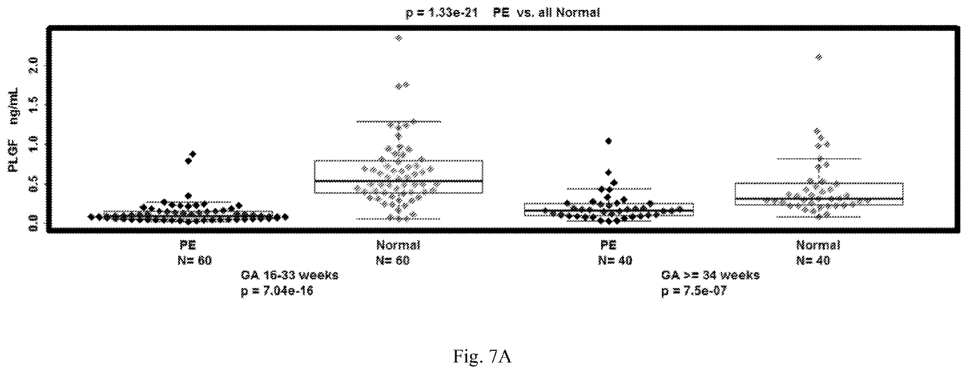

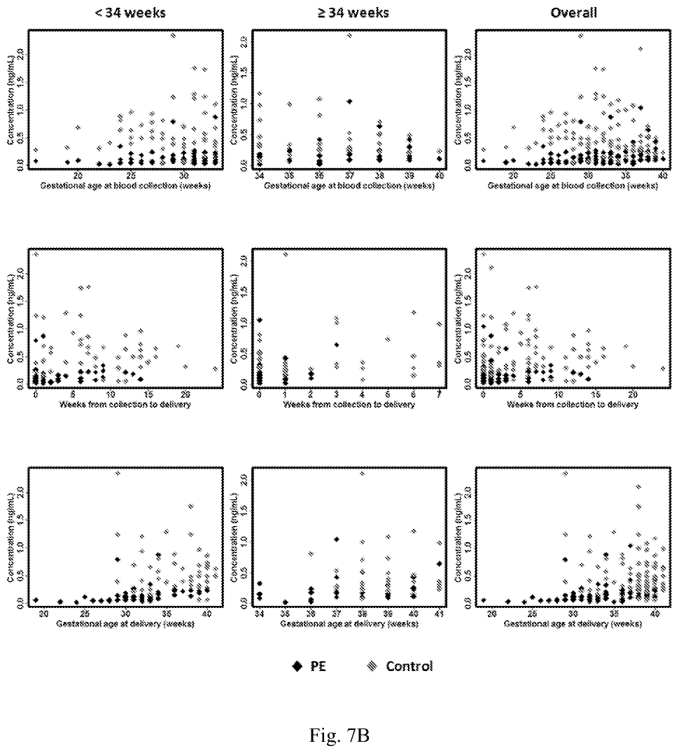

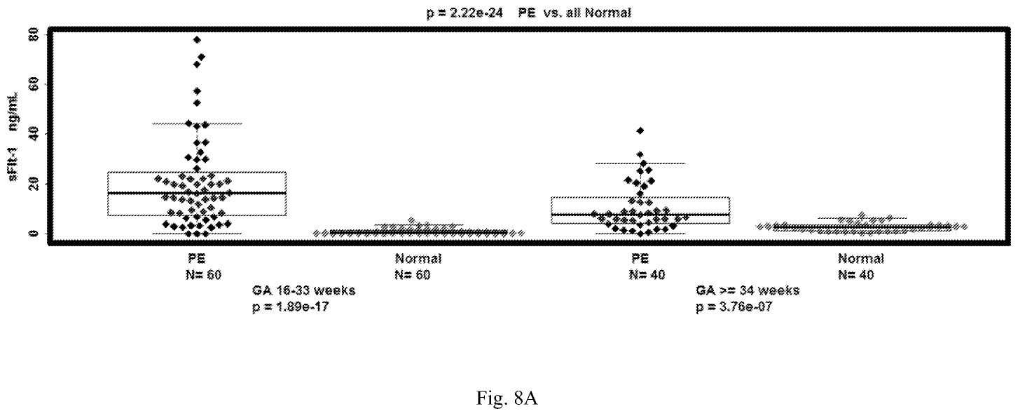

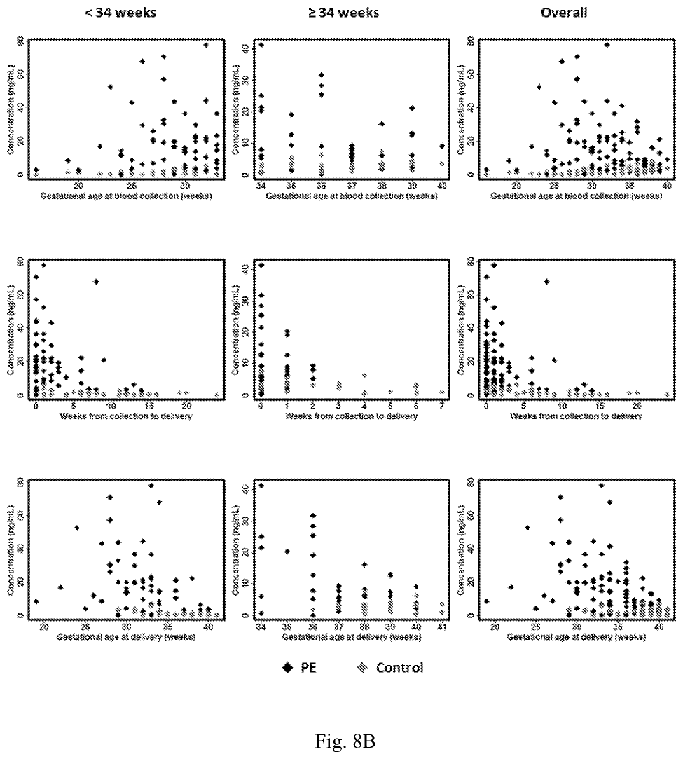

To identify whether the PE serological protein panel could enable development of an immediate practical clinical tool, based on available ELISA assays, biomarker candidates, from expression meta-analysis and 2D gel profiling, were validated with available serum assays using PE (n=100) and gestation age-matched control samples (n=100). Detailed with whisker box and scatter plots in FIGS. 4-8, five proteins were validated by ELISA assays (Mann-Whitney U-test). FIGS. 4-8 also demonstrated the distribution of maternal serum abundance of each validated protein over the gestational age (weeks) of blood sample collection, delivery, and the gap in between. Each validated biomarker's median, mean and standard deviation of maternal serum abundance, in PE and control samples, are summarized in Table 11.

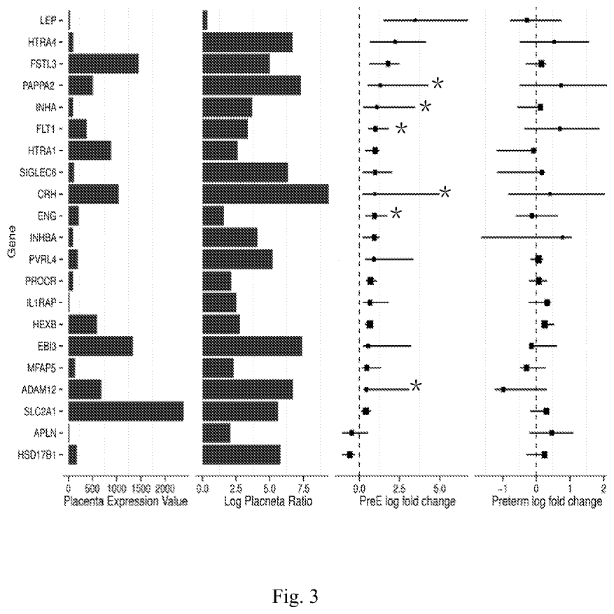

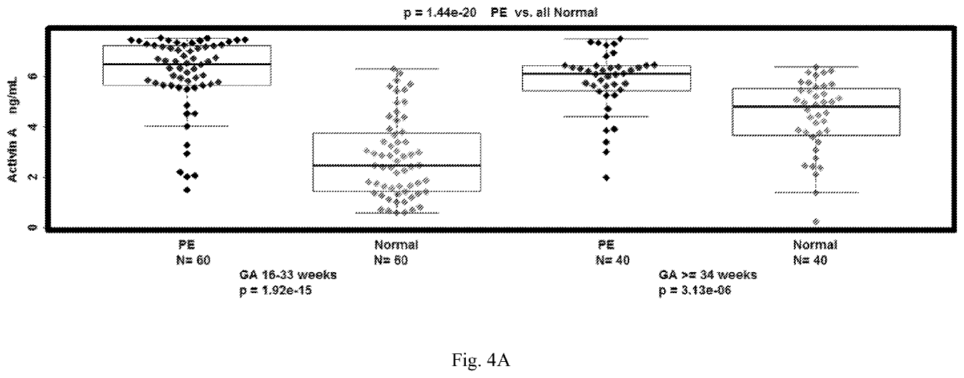

Forest plots (FIG. 3) summarize the PE to control ratios of 21 PE markers across placental expression meta-analyses, and early and late gestation maternal serum analyses. The biomarkers derived from the proteomic and expression meta-analyses consistently shared the same trend of up- or down-regulation between PE and control samples.

Univariate and Multivariate Analysis of Validated PE Biomarkers.

Univariate and multivariate analyses were performed on each of the five validated PE biomarkers (Tables 12-19). Results of odds ratios and hazard ratios in univariate analysis showed that all five biomarkers (except EPCR at late stage) had significant impact (p<0.05) on preeclampsia. Multivariate analysis of hazard ratio showed that Activin A and PIGF had significant impact (p<0.05), indicating panels consisting of these biomarkers may achieve optimum classification performance of PE and control subjects.

PE Biomarker Panel Construction.

Using data from the ELISA assays, we constructed different panels with various subsets of the assays (Table 20). We sought to identify biomarker panels of optimal feature number, balancing the need for small panel size, accuracy of classification, goodness of class separation (PE versus control), and sufficient sensitivity and specificity. With the aim to develop a multiplexed antibody-based assay for PE diagnosis, we used a geometric mean method to construct biomarker panels from the 5 validated PE protein biomarkers for early and late gestational age PE, comparing to the ratio of sFlt-1 to PIGF in assessing PE. These chosen biomarker panels are non-redundant, indicating non-inclusive relationships. The sFlt-1/PIGF ratio's PE assessment utility (early onset, receiver operating characteristics curve ROC area under the curve 0.9581, p value 2.52×10⁻¹⁸; late onset, ROC AUC 0.8288, p value 5.09×10⁻⁸; overall, ROC AUC 0.9284, p value 6.17×10⁻²⁸), previously through the multicenter trial validation (Verlohren et al. An automated method for the determination of the sFlt-1/PIGF ratio in the assessment of preeclampsia. American journal of obstetrics and gynecology 2010; 202:161 e1-61 e11), was confirmed in this study and used as a benchmark for our newly derived biomarker panels.

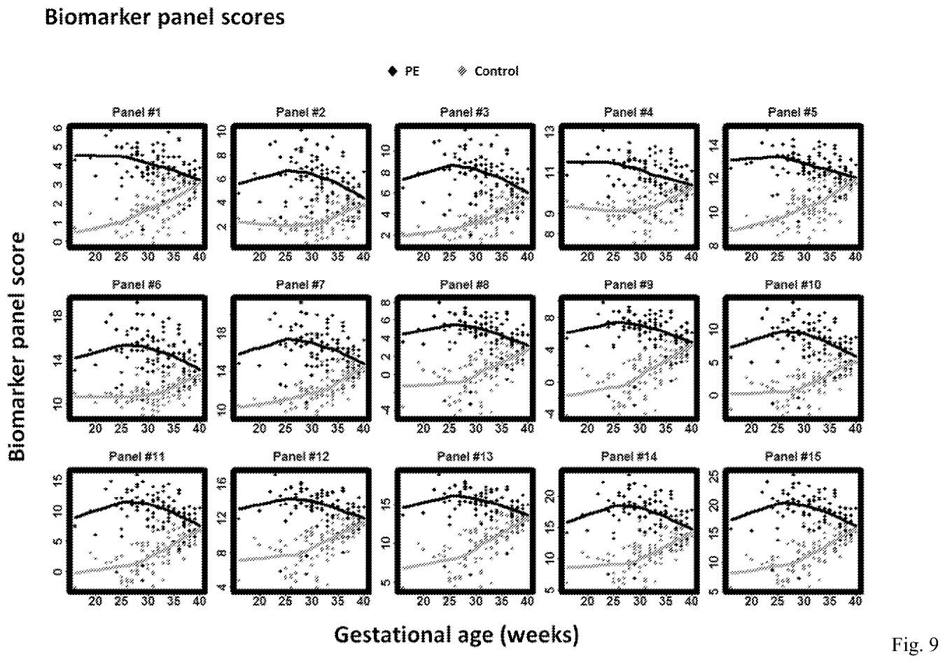

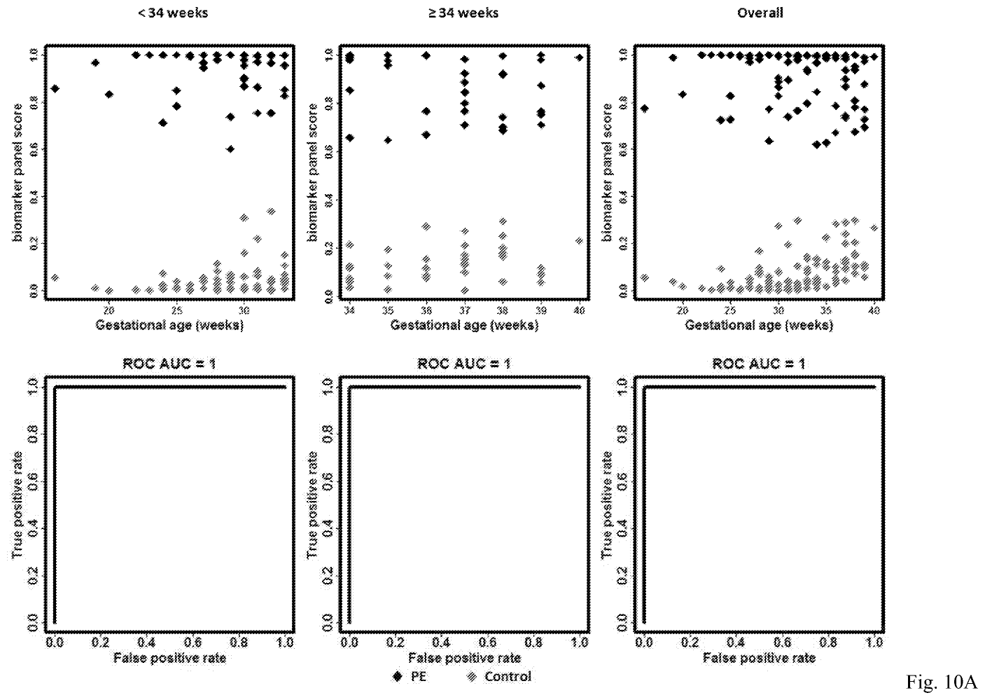

To demonstrate the efficacy of the biomarker panel as a classifier for PE disease activity according to disease onset, the biomarker panel scores were plotted as a function of time of the gestational age (details shown in FIG. 9). ROC AUC performance for each panel was shown on Table 21. According to the scatter plot analysis and ROC AUC analysis, there are 8 panels (Panel 1, 3, 5, 6, 7, 9, 13, 15) having ROC AUC higher than sFlt-1/PIGF at all stages (early, late, and overall). Panel 1 has two proteins; Panel 3, 5, 6, and 9 have three proteins; Panel 7 and 13 have four proteins; Panel 15 have 5 proteins. For gestational age <34 weeks subjects, 10 panels (Panels 1-3, 5-7, 9, 12, 13, and 15) had better ROC AUC performance than the sFlt-1/PIGF ratio on early stage (gestational age <34 weeks). For gestational age 34 weeks subjects, all of our panels except Panels 4 and 12 had better ROC AUC performance than the sFlt-1/PIGF. For the overall ROC AUC performance, Panels 1, 5 and 7 were three having the largest AUC values (Panel 1, 0.9397, p value 3.22×10⁻²⁷; Panel 5, 0.9419, p value 1.80×10⁻²⁷; Panel 7, 0.9394, p value 3.48×10⁻²⁷). Activin A are present in all these three panels, suggesting its critical role in the diagnosis and perhaps pathophysiology of PE.

Pathway Analysis of PE Biomarkers.

We analyzed the validated biomarkers that are significantly differentially expressed in PE as a composite, using PathVisio software (version 3.2.1, an open-source pathway analysis and drawing software) (Martijn et al. Presenting and exploring biological pathways with PathVisio. BMC Bioinformatics 2008; 9 (1): 399). In addition to the angiogenesis and focal adhesion pathway involved well-studied PE biomarker FLT1, our pathway analysis led to the identification of the following statistically significant canonical pathways which may play important roles in PE pathophysiology: TGF-beta signaling pathway; differentiation pathway; senescence and autophagy; Inhibition of Matrix Metalloproteinases; the protein C pathway and Regulation of Insulin-like Growth Factor (IGF) Transport and Uptake by Insulin-like Growth Factor Binding Proteins (IGFBPs). The TGF-beta signaling pathway was identified as the most significant pathway. This supports recent findings (Yong et al. Effects of normal and high circulating concentrations of Activin A on vascular endothelial cell functions and vasoactive factor production. Pregnancy Hypertens 2015; 5(4):346-53) and our hypothesis that PE is associated with increased shedding of placental debris leading to increased plasma levels of TGF proteins, which could contribute to the inflammatory response and endothelial dysfunction.

### Discussion

We have applied a multi-‘omics’ approach to develop validated PE biomarkers, integrating discoveries from placental mRNA expression meta-analysis and depleted serological proteome 2D gel comparative profiling. Comparing PE and control sera with commercially available ELISA assays, we have validated 5 protein markers, including sFlt-1 and PIGF, and found that our identified PE biomarkers were comparable to sFlt-1/PIGF ratio in predicting PE. The concept of combining a transcriptomic approach in placenta tissue with a proteomic approach in serum is novel. It combines the advantages of a study in tissue which is closer to the focus of the pathophysiology with those of a study in serum which is more appropriate for clinical use. Taking proteins that have been discovered/predicted from the discovery phase to an ELISA-based validation phase makes the findings of this study translatable into clinical practice.

Pathogenesis of Preeclampsia has been studied for years. Although the cause is unknown, trophoblast dysfunction has been most widely accepted as the key pathophysiological changes during preeclampsia (Redman, 1991). Releasement of various factors from trophoblasts trigger inflammatory responses and result in maternal symptoms such as hypertension, proteinuria, edema, as well as other organ dysfunctions. It has been established that the main source of circulating Activin A is from foeto-placental unit during first trimester pregnancy (Muttukrishna et al., 1997). Activin A can stimulate FSH biosynthesis as well as secretion (Muttukrishna and Knight, 1991), and it regulates trophoblast cell differentiation (Caniggia et al., 1997). Activin receptor type 2A (ACVR2A) polymorphisms have been suggested as the genetic risk factor for preeclampsia (Fitzpatrick et al., 2009; Roten et al., 2009). The oxidative stress induced during trophoblast dysfunction can increase Activin A secretion (Mandang et al., 2007). Circulating Activin A has been proposed to predict preeclampsia when combining with uterine artery Doppler ultrasonography (Spencer et al., 2006).

The involvement of pro- and anti-angiogenesis has also been extensively studied in preeclasmpsia (Levine et al., 2004). sFlt-1 and soluble Endoglin (sENG) are two anti-angiogenic factors. sFlt-1 is also known as soluble VEGF receptor I, it correlates with the severity as well as the time onset of preeclampsia (Maynard et al., 2003). Soluble Endoglin is highly expressed in maternal endothelial cells and trophoblasts, and it's a co-receptor for TGF-β1&β3 (Gu et al., 2008). PIGF is a placental pro-angiogenic factor and potentiates the effect of VEGF (Levine et al., 2006). Placental hypoxia and subsequent oxidative stress increases the expression of sFlt-1 and sENG levels in the extravillous trophoblasts, and those anti-angiogenic factors inhibits trophoblast autophagy, which is essential for trophoblast invasion and vascular remodeling during placentation (Nakashima et al., 2013). sFlt-1/PIGF ratio has been proposed as a predictive marker for preeclampsia (Levine et al., 2004; Zeisler et al., 2016).

PAPPA2 is a large protein complex expressed by trophoblasts (Bersinger et al., 2003). It has been reported that downregulation of PAPPA2 has been associated with early onset preeclampsia (Smith et al., 2002). Although itself is not a specific biomarker for preeclampsia (Canini et al., 2008), it does have predict value when combining with uterine artery Doppler ultrasonography (Spencer et al., 2008).

A previous multicenter case-control study (Verlohren et al. An automated method for the determination of the sFlt-1/PIGF ratio in the assessment of preeclampsia. American journal of obstetrics and gynecology 2010; 202:161 e1-61 e11) with an automated assay, demonstrating the utilities of sFlt-1 and PIGF for PE assessment, reported serum abundance of sFlt-1 (PE: 12,981±965 vs control: 2641±100.5 pg/mL) and PIGF (PE: 76.06±10.71 vs control: 341.5±13.57 pg/mL). Although with greater variation, possibly due to different sample cohorts or assay platforms, the trend of alteration reflected in our results, sFlt-1 (PE: 16,470±15,740 vs control: 1660±1665 pg/mL) and PIGF (PE: 164.01±169.05 vs control: 564.09±417.97 pg/mL) was in line with their report. As shown in FIGS. 4-8 and summarized in Table 22 (below), protein abundance of all our biomarkers differs significantly (p value<0.05) between early and late gestational age samples in normal group. In PE group, Activin A and EPCR (Table 22) were not significantly (p value>0.05) different between early and late gestation age. Our results here indicate that PIGF, sFlt-1, and ENG are regulated during placental development as a function of gestation in control, and differential expression between PE and control might be due to placental adaptation during PE. The other PE biomarkers found in this study (Activin A and EPCR) are not significantly different between early and late gestation in PE sera. Therefore, their differential expression in PE might directly gauge the pathogenesis of PE and disease development or reflect features that are present at fairly advanced stages of the pathogenesis, e.g. proteinuria and high blood pressure, which are not necessarily related to its pathophysiology.

Our genetic algorithm-based biomarker panel construction led to final early and late gestational age biomarker panels for PE assessment. Compared to the benchmark sFlt-1/PIGF ratio in PE assessment, our biomarker panels clearly outperform at both early and late gestation age stage. Although the sFlt-1 and PIGF imbalance used for PE diagnosis has been demonstrated, there is mounting evidence to support the notion that normal sFlt-1 and PIGF expression actually characterizes healthy pregnancies (Daponte et al. Soluble fms-like tyrosine kinase-1 (sFlt-1) and serum placental growth factor (PIGF) as biomarkers for ectopic pregnancy and missed abortion. The Journal of clinical endocrinology and metabolism. 2011; 96:E1444-1451). Therefore, sFlt-1 and PIGF may really be general markers for failed pregnancies, e.g. ectopic pregnancies, missed abortions, rather than specific to PE. Our multi-‘omics’ approach discovered panels of multiple biomarkers, reflecting the multifaceted aspects of PE pathophysiology, and have the potential to provide a definitive diagnosis of PE patients, to identify patients at risk, and to be used to monitor disease progression.

### Example 2

The protein levels of panels of preeclampsia markers described in Example 1 and 2 were assayed in serum of preeclampsia patients to determine the accuracy of these additional panels in diagnosing early onset preeclampsia (e.g. onset of preeclampsia prior to 34 weeks of gestation) or late onset preeclampsia (i.e. onset of preeclampsia at 34 weeks of gestation or later). Panels of particular interest were the following (see Table 20):
Panel 1: Activin A, PIGF
Panel 2: ENG, PIGF
Panel 3: Activin A, ENG, PIGF
Panel 4: EPCR, PIGF
Panel 5: ActivinA, EPCR, PIGF
Panel 6: ENG, EPCR, PIGF
Panel 7: Activin A, ENG, EPCR, PIGF
Panel 8: sFlt-1, PIGF
Panel 9: Activin A, sFlt-1, PIGF
Panel 10: ENG, sFlt-1, PIGF
Panel 11: Activin A, ENG, sFlt-1, PIGF
Panel 12: EPCR, sFlt-1, PIGF
Panel 13: Activin A, EPCR, sFlt-1, PIGF
Panel 14: ENG, EPCR, sFlt-1, PIGF
Panel 15: Activin A, ENG, EPCR, sFlt-1, PIGF

Panel 8 comprises markers that form the current standard for diagnosing preeclampsia. Other panels comprise markers in Panel 8 and additional preeclampsia markers disclosed herein.

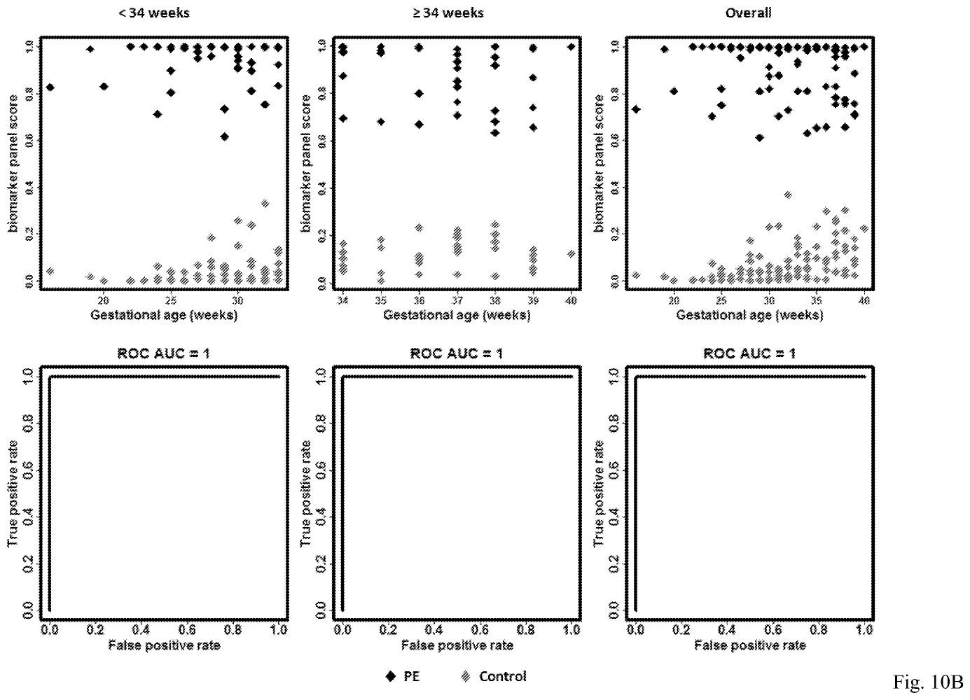

As illustrated in Table 23, all the panels had better or comparable sensitivity and comparable specificity than the current standard for diagnosing preeclampsia at early stage and late stage. Sensitivity values at different specificity levels and specificity values at different sensitivity levels were shown in Tables 24 and 25. Indeed, FIGS. 10 A and B showed that a panel comprising all five validated preeclampsia markers disclosed herein (Activin A, ENG, EPCR, PIGF, and sFlt-1) provide 100% accuracy in diagnosing preeclampsia at their designated time (AUC=1 at early, late, and overall stage).

### Example 3

The protein levels of a panel of preeclampsia markers (Activin A, ENG, EPCR, PIGF, and sFlt-1) was statistically assessed to determine how to weigh the contribution of each polypeptide to a preeclampsia score for a sample based on this panel.

Using the random forest algorithm, EPCR levels were determined to be least significant; Activin A levels were determined to be about 1.2-fold more significant than EPCR; ENG and PIGF levels were determined to be about 1.6-fold more significant that EPCR; and sFlt-1 levels were determined to be most significant, i.e. about 2.3-fold more significant than EPCR (see Table 26).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

### Tables

TABLE 1
The microarray datasets studied in present work.

| | | | | Gestational age | | | |
|---|---|---|---|---|---|---|---|
| | PE case | Gestational age of | Control | of case v.s. | | | |
| No. GEO | (n =) | case | (n =) | control | Dissection | Year and Authors | PMID |
| GSE4707 | 14 | <31 wks (n = 6) | 13 | matched | placental | Nishizawa H, | 16860862 |
| | | (early) ≥31 wks | | | vili | Pryor-Koishi K, Kato | |
| | | (n = 8) (late) | | | | T, et al., 2007 | |
| GSE10588 | 21 | <34 wks | 21 | matched | chorionic | Sitras V, Paulssen | 19249095 |
| | | (early) >34 wks | | | villi | RH, Grønaas H, et al., | |
| | | (late) | | | | 2009 | |
| GSE24129 | 8 | multiple gestation | 8 | multiple | chorionic | Nishizawa H, Ota S, | 21810232 |
| | | | | gestation | villi | Suzuki M, et al., 2011 | |
| GSE25906 | 23 | 27-38 wks | 37 | 29-40 wks | placental | Tsai S, Hardison NE, | 21183218 |
| | | | | | vili | James AH, et al., | |
| | | | | | | 2011 | |
| GSE44711 | 20 | 8 early, 12 late | 20 | 8 early, 12 late | chorionic | Blair JD, Yuen RK, von | 23770704 |
| | | | | | villi | Dadelszen P, et al., | |
| | | | | | | 2013 | |
| GSE54618 | 12 | 26-38 wks | 12 | 26-40 wks | placental | Jebbink JM, Boot RG, | 25552189 |
| | | | | | vili | Ris-Stalpers C, 2015 | |
| Total | 98 | | 111 | | | | |

TABLE 2
Profiles of candidates of PE biomarkers.

| | | | | | | | Gene original from | | |
|---|---|---|---|---|---|---|---|---|---|
| | | | | | | | | | Genetic |
| | | | | | | | | | defect |
| | PE. Differ. | | | | expression | | | PE. | human |
| Gene | fold | | | | ratio in | ELISA | Placenta | Differential | orthologous |
| Symbol | change | p-value | regulation | Circulation | placenta | kit | genes | genes | genes |
| FLT1 | 1.87 | 6.92E−05 | [symbol] | + | 3.37 | Yes | + | + | + |
| PROCR | 1.53 | 4.08E−05 | [symbol] | − | 2.16 | Yes | | + | + |
| ENG | 1.64 | 0.00043647 | [symbol] | + | 1.60 | Yes | | + | + |
| PAPPA2 | 2.07 | 1.94E−05 | [symbol] | + | 7.31 | Yes | + | + | − |
| INHBA | 1.53 | 1.81E−05 | [symbol] | − | 4.09 | Yes | + | + | − |
| ADAM12 | 1.34 | 0.001239043 | [symbol] | + | 6.72 | Yes | + | + | − |
| FSTL3 | 2.47 | 0.000259531 | [symbol] | − | 4.99 | Yes | + | + | − |
| APLN | −1.3 | 0.00060869 | [symbol] | − | 2.08 | Yes | + | + | − |
| LEP | 6.87 | 8.89E−08 | [symbol] | − | 0.36 | Yes | + | + | − |
| INHA | 1.76 | 1.40E−05 | [symbol] | + | 3.71 | No | + | + | − |
| PIK3CB | 1.37 | 0.003393934 | [symbol] | − | 1.13 | No | | + | + |
| SLC2A1 | 1.33 | 0.000255123 | [symbol] | − | 5.61 | No | + | + | − |
| CRH | 2.11 | 0.001342342 | [symbol] | + | 9.79 | No | + | + | − |
| HSD17B1 | −1.47 | 0.001968052 | [symbol] | − | 5.79 | No | + | + | − |
| SIGLEC6 | 1.49 | 3.19E−05 | [symbol] | − | 6.34 | No | + | + | − |
| PVRL4 | 1.66 | 3.33E−05 | [symbol] | − | 5.21 | No | + | + | − |
| HEXB | 1.43 | 0.000444465 | [symbol] | − | 2.79 | No | + | + | − |
| IL1RAP | 1.43 | 0.000557809 | [symbol] | − | 2.52 | No | + | + | − |
| MFAP5 | 1.37 | 0.001158913 | [symbol] | − | 2.32 | No | + | + | − |
| HTRA1 | 1.55 | 0.000303748 | [symbol] | − | 2.63 | No | + | + | − |
| EBI3 | 1.54 | 0.00070665 | [symbol] | − | 7.40 | No | + | + | − |
| HTRA4 | 2.82 | 3.12E−05 | [symbol] | − | 6.68 | No | + | + | − |

TABLE 3
Enrolled pregnant subjects with blood collected at <34 weeks and ≥34 weeks.

| | <34 weeks | | | | | | |
|---|---|---|---|---|---|---|---|
| | PE | Control | | ≥34 weeks | | | |
| | N = 60 | N = 60 | | PE | Control | | Overall |
| Characteristic | (50%) | (50%) | P value | N = 40 (50%) | N = 40 (50%) | P value | P value |
| Ethnicity, n | | | 0.99^{a} | | | | 0.99^{a} |
| (%) | | | | | | | |
| Han | 59 (98.3) | 59 (98.3) | | 40 (100) | 40 (100) | | |
| Manchu | 1 (1.7) | 1 (1.7) | | 0 | 0 | | |
| Age (year) | | | | | | | |
| mean (SD) | 29.6 (5.7) | 28.7 (5.1) | 0.38^{b} | 29.6 (5.6) | 29.9 (4.7) | 0.81^{b} | 0.59^{b} |
| Week of | | | | | | | |
| gestation at | | | | | | | |
| blood | | | | | | | |
| collection | | | | | | | |
| mean (SD) | 28.6 (3.8) | 28.6 (3.8) | 0.99^{b} | 36.6 (1.8) | 36.6 (1.8) | 0.99^{b} | 0.99^{b} |
| Week of | | | | | | | |
| gestation | | | | | | | |
| at delivery | | | | | | | |
| mean (SD) | 31.5 (4.1) | 36.6 (3.6) | <0.001^{b} | 37.2 (1.9) | 38.6 (1.4) | <0.001^{b} | <0.001^{b} |
| Weeks from | | | | | | | |
| blood | | | | | | | |
| collection to | | | | | | | |
| delivery | | | | | | | |
| mean (SD) | 2.7 (3.7) | 8.1 (6.1) | <0.001^{b} | 0.6 (0.8) | 2.1 (2.5) | <0.001^{b} | <0.001^{b} |

^{a}Fisher's exact test;
^{b}Student's t-test.

TABLE 4
Concurrent medical conditions and clinical features
of the enrolled case and control subjects.

| | PE | Control | |
|---|---|---|---|
| Characteristic | N = 100 (50%) | N = 100 (50%) | P value |
| Concurrent medical | | | 0.061^{a} |
| conditions/Clinical features | | | |
| Hyperthyroidism | 3 (3%) | 0 (0%) | |
| Diabetes-type II | 0 (0%) | 1 (1%) | |
| Diabetes (gestational) | 10 (10%) | 12 (12%) | |
| Fatty liver | 3 (3%) | 0 (0%) | |
| Hypothyroidism | 1 (1%) | 1 (1%) | |
| Intrahepatic cholestasis | 1 (1%) | 2 (2%) | |
| Preterm birth | 0 (0%) | 7 (7%) | |
| Raynaud's syndrome | 0 (0%) | 1 (1%) | |
| Hashimoto's Thyroiditis | 0 (0%) | 1 (1%) | |
| Nephrotic syndrome | 1 (1%) | 0 (0%) | |
| Kaliopenia | 1 (1%) | 0 (0%) | |
| Hyperproteinemia | 1 (1%) | 0 (0%) | |
| Subclinical hypothyroidism | 1 (1%) | 0 (0%) | |
| Anemia | 0 (0%) | 1 (1%) | |
| Pregnancy induced | 0 (0%) | 1 (1%) | |
| hypertension | | | |
| None | 47 (47%) | 49 (49%) | |
| Unknown | 31 (31%) | 26 (26%) | |

^{a}Fisher's exact test.

TABLE 5
PE patients' presenting signs and symptoms.

| Presenting signs and symptoms | Number (percentage) |
|---|---|
| Hypertension | 100 (100%) |
| Proteinuria | 91 (91%) |
| Headache | 12 (12%) |
| Edema | 59 (59%) |
| Others/unknown | 4 (4%) |

TABLE 6
Clinical information of the enrolled case and control subjects. Clinical information was
unavailable for one control subject.

| | PE | | Control | | |
|---|---|---|---|---|---|
| Characteristics | N = 100 | | N = 100 | | P value |
| BMI (prior to pregnancy) (kg/m²), median (IQR) | 21.6 | (20.0, 23.3) | 20.3 | (18.9, 22.0) | 0.002^{a} |
| Systolic blood pressure, median (IQR) | 158.5 | (147.0, 170.0) | 118.0 | (110, 122.5) | <0.001^{a} |
| Diastolic blood pressure, median (IQR) | 101.5 | (93.0, 109.0) | 71.0 | (69.0, 78.0) | <0.001^{a} |
| Protein/creatinine ratio (PCR) test results (mg/g), | 1033.0 | (731.0, 1292.6) | NA | | |
| median (IQR) | | | | | |
| Prior history of preeclampsia, n (%) | | | | | 0.003^{b} |
| Yes | 9 | (9) | 0 | | |
| Multiple gestation, n (%) | | | | | 0.33^{b} |
| Yes | 3 | (3) | 6 | (6) | |
| Number of abortions, n (%) | | | | | 0.13^{b} |
| number = 0 | 44 | (44) | 58 | (59) | |
| number = 1 | 32 | (32) | 26 | (26) | |
| number = 2 | 14 | (14) | 11 | (11) | |
| number = 3 | 8 | (8) | 2 | (2) | |
| number = 4 | 2 | (2) | 1 | (1) | |
| number = 5 | 0 | | 1 | (1) | |
| Number of full term pregnancies, n (%) | | | | | 0.69^{b} |
| number = 0 | 73 | (73) | 67 | (68) | |
| number = 1 | 24 | (24) | 30 | (30) | |
| number = 2 | 2 | (2) | 2 | (2) | |
| number = 3 | 1 | (1) | 0 | | |
| Number of premature pregnancies, n (%) | | | | | 0.059^{b} |
| number = 0 | 95 | (95) | 99 | (100) | |
| number = 1 | 5 | (5) | 0 | | |
| Smoking history, n (%) | | | | | 0.99^{b} |
| Never | 99 | (99) | 99 | (100) | |
| Total number of pregnancies, n (%) | | | | | 0.41^{b} |
| number = 1 | 35 | (35) | 49 | (50) | |
| number = 2 | 30 | (30) | 25 | (25) | |
| number = 3 | 16 | (16) | 14 | (14) | |
| number = 4 | 13 | (13) | 8 | (8) | |
| number = 5 | 4 | (4) | 2 | (2) | |
| number = 6 | 1 | (1) | 0 | | |
| number = 7 | 1 | (1) | 1 | (1) | |
| In vitro fertilization (IVF) utilized for this | | | | | 0.99^{b} |
| pregnancy, n (%) | | | | | |
| No | 97 | (97) | 96 | (97) | |
| Maternal height, cm, mean (SD) | | | | | 0.189^{a} |
| Maternal weight, kg, mean (SD) | 56.2 | (8.7) | 53.2 | (6.5) | 0.005^{a} |
| Delivery mode, n (%) | 159.6 | (4.6) | 160.6 | (4.3) | <0.001^{c} |
| Eutocia | 8 | (8) | 40 | (40.8) | |
| Caesarean | 66 | (66) | 47 | (48) | |
| Other | 26 | (26) | 11 | (11.2) | |
| Fetus height (cm), median, IQR | 43 | (38, 49) | 50 | (48.2, 50) | <0.001^{a} |
| Fetus weight (g), mean (SD) | 1907.4 | (950.1) | 3024.3 | (741.7) | <0.001^{a} |
| Proteinuria, N (%) | 91 | (93.8) | 5 | (6.2) | <0.001^{c} |

^{a}Ranksum test;
^{b}Fisher's exact test;
^{c}Chi-squared test.

Table 7. Univariate odds ratio analysis of the patient characteristics.

7A. Enrolled subjects blood collected <34 weeks of gestational age;
results were achieved without adjustment of gestational age at blood
sample collection.

| Characteristics | OR | 95CI (L) | 95CI (H) | P value |
|---|---|---|---|---|
| BMI prior to pregnancy (kg/m²) | 1.25 | 1.07 | 1.49 | 0.0076 |
| Age (year) | 1.03 | 0.96 | 1.1 | 0.3785 |
| Concurrent diabetes during | 1 | 0.3 | 3.38 | 1 |
| pregnancy | | | | |

7B. Enrolled subjects blood collected ≥34 weeks of gestational age;
results were achieved without adjustment of gestational age at blood
sample collection.

| Characteristics | OR | 95CI (L) | 95CI (H) | P value |
|---|---|---|---|---|
| BMI prior to pregnancy (kg/m²) | 1.13 | 0.99 | 1.32 | 0.0806 |
| Age (year) | 0.99 | 0.91 | 1.08 | 0.81 |
| Concurrent diabetes during | 1.59 | 0.42 | 6.67 | 0.5015 |
| pregnancy | | | | |

7C. All enrolled subjects; results were achieved without adjustment
of gestational age at blood sample collection.

| Characteristics | OR | 95CI (L) | 95CI (H) | P value |
|---|---|---|---|---|
| BMI prior to pregnancy (kg/m²) | 1.18 | 1.07 | 1.33 | 0.0022 |
| Age (year) | 1.01 | 0.96 | 1.07 | 0.5863 |
| Concurrent diabetes during | 1.23 | 0.5 | 3.05 | 0.6517 |
| pregnancy | | | | |

7D. Enrolled subjects blood collected <34 weeks of gestational age;
results were achieved with adjustment of gestational age at blood
sample collection.

| Characteristics | OR | 95CI (L) | 95CI (H) | P value |
|---|---|---|---|---|
| BMI prior to pregnancy (kg/m²) | 1.25 | 1.07 | 1.49 | 0.0074 |
| Age (year) | 1.03 | 0.96 | 1.11 | 0.3684 |
| Concurrent diabetes during | 1 | 0.3 | 3.39 | 1 |
| pregnancy | | | | |

7E. Enrolled subjects blood collected ≥34 weeks of gestational age;
results were achieved with adjustment of gestational age at blood
sample collection.

| Characteristics | OR | 95CI (L) | 95CI (H) | P value |
|---|---|---|---|---|
| BMI prior to pregnancy (kg/m²) | 1.14 | 0.99 | 1.33 | 0.0785 |
| Age (year) | 0.99 | 0.91 | 1.08 | 0.8093 |
| Concurrent diabetes during | 1.65 | 0.41 | 7.3 | 0.4834 |
| pregnancy | | | | |

7F. All enrolled subjects; results were achieved with adjustment of
gestational age at blood sample collection.

| | | 95CI | | |
|---|---|---|---|---|
| Characteristics | OR | (L) | 95CI (H) | P value |
| BMI prior to pregnancy (kg/m²) | 1.18 | 1.07 | 1.33 | 0.0022 |
| Age (year) | 1.01 | 0.96 | 1.07 | 0.5875 |
| Concurrent diabetes during pregnancy | 1.23 | 0.5 | 3.05 | 0.6511 |

Table 8. Multivariate odds ratio analysis of the patient characteristics.

8A. Enrolled subjects blood collected < 34 weeks of gestational age;
results were achieved without adjustment of gestational age at blood
sample collection.

| | | 95CI | | |
|---|---|---|---|---|
| Characteristics | OR | (L) | 95CI (H) | P value |
| BMI prior to pregnancy (kg/m²) | 1.25 | 1.06 | 1.5 | 0.0103 |
| Age (year) | 1.01 | 0.94 | 1.09 | 0.8017 |
| Concurrent diabetes during pregnancy | 0.74 | 0.21 | 2.62 | 0.638 |

8B. Enrolled subjects blood collected ≥ 34 weeks of gestational age;
results were achieved without adjustment of gestational age at blood
sample collection.

| | | 95CI | | |
|---|---|---|---|---|
| Characteristics | OR | (L) | 95CI (H) | P value |
| BMI prior to pregnancy (kg/m²) | 1.14 | 0.99 | 1.32 | 0.0776 |
| Age (year) | 0.98 | 0.89 | 1.07 | 0.6403 |
| Concurrent diabetes during pregnancy | 1.58 | 0.4 | 6.79 | 0.5155 |

8C. All enrolled subjects; results were achieved without adjustment of
gestational age at blood sample collection.

| | | 95CI | | |
|---|---|---|---|---|
| Characteristics | OR | (L) | 95CI (H) | P value |
| BMI prior to pregnancy (kg/m²) | 1.18 | 1.07 | 1.33 | 0.0027 |
| Age (year) | 1 | 0.94 | 1.06 | 0.9636 |
| Concurrent diabetes during pregnancy | 1.07 | 0.43 | 2.72 | 0.8895 |

8D. Enrolled subjects blood collected < 34 weeks of gestational age;
results were achieved with adjustment of gestational age at blood
sample collection.

| | | 95CI | | |
|---|---|---|---|---|
| Characteristics | OR | (L) | 95CI (H) | P value |
| BMI prior to pregnancy (kg/m²) | 1.25 | 1.06 | 1.5 | 0.0103 |
| Age (year) | 1.01 | 0.94 | 1.09 | 0.7648 |
| Concurrent diabetes during pregnancy | 0.74 | 0.21 | 2.61 | 0.6349 |

8E. Enrolled subjects blood collected ≥ 34 weeks of gestational age;
results were achieved with adjustment of gestational age at blood
sample collection.

| | | 95CI | | |
|---|---|---|---|---|
| Characteristics | OR | (L) | 95CI (H) | P value |
| BMI prior to pregnancy (kg/m²) | 1.14 | 0.99 | 1.33 | 0.0792 |
| Age (year) | 0.98 | 0.89 | 1.07 | 0.6336 |
| Concurrent diabetes during pregnancy | 1.54 | 0.37 | 6.94 | 0.5563 |

8F. All enrolled subjects; results were achieved with adjustment of
gestational age at blood sample collection.

| | | 95CI | | |
|---|---|---|---|---|
| Characteristics | OR | (L) | 95CI (H) | P value |
| BMI prior to pregnancy (kg/m²) | 1.18 | 1.07 | 1.33 | 0.0027 |
| Age (year) | 1 | 0.94 | 1.06 | 0.9609 |
| Concurrent diabetes during pregnancy | 1.07 | 0.43 | 2.73 | 0.8865 |

Table 9. Univariate hazard ratio analysis of the patient characteristics.

9A. Enrolled subjects blood collected < 34 weeks of gestational age;
results were achieved without adjustment of gestational age at blood
sample collection.

| | | 95CI | | |
|---|---|---|---|---|
| Characteristics | HR | (L) | 95CI (H) | P value |
| BMI prior to pregnancy (kg/m²) | 1.12 | 1.02 | 1.23 | 0.015 |
| Age (year) | 1.02 | 0.97 | 1.07 | 0.4015 |
| Concurrent diabetes during pregnancy | 0.87 | 0.37 | 2.01 | 0.7373 |

9B. Enrolled subjects blood collected ≥ 34 weeks of gestational age;
results were achieved without adjustment of gestational age at blood
sample collection.

| | | 95CI | | |
|---|---|---|---|---|
| Characteristics | HR | (L) | 95CI (H) | P value |
| BMI prior to pregnancy (kg/m²) | 1.09 | 1 | 1.18 | 0.0439 |
| Age (year) | 0.99 | 0.93 | 1.06 | 0.8273 |
| Concurrent diabetes during pregnancy | 1.1 | 0.46 | 2.62 | 0.8336 |

9C. All enrolled subjects; results were achieved without adjustment of
gestational age at blood sample collection.

| | | 95CI | | |
|---|---|---|---|---|
| Characteristics | HR | (L) | 95CI (H) | P value |
| BMI prior to pregnancy (kg/m²) | 1.08 | 1.02 | 1.14 | 0.0126 |
| Age (year) | 1.01 | 0.97 | 1.05 | 0.7629 |
| Concurrent diabetes during pregnancy | 0.98 | 0.54 | 1.8 | 0.9537 |

9D. Enrolled subjects blood collected < 34 weeks of gestational age;
results were achieved with adjustment of gestational age at blood
sample collection.

| | | 95CI | | |
|---|---|---|---|---|
| Characteristics | HR | (L) | 95CI (H) | P value |
| BMI prior to pregnancy (kg/m²) | 1.12 | 1.02 | 1.23 | 0.0132 |
| Age (year) | 1.01 | 0.96 | 1.06 | 0.6556 |
| Concurrent diabetes during pregnancy | 0.87 | 0.38 | 2.03 | 0.7561 |

9E. Enrolled subjects blood collected ≥ 34 weeks of gestational age;
results were achieved with adjustment of gestational age at blood
sample collection.

| | | 95CI | | |
|---|---|---|---|---|
| Characteristics | HR | (L) | 95CI (H) | P value |
| BMI prior to pregnancy (kg/m²) | 1.09 | 1 | 1.19 | 0.0475 |
| Age (year) | 1 | 0.94 | 1.06 | 0.9669 |
| Concurrent diabetes during pregnancy | 1.28 | 0.51 | 3.22 | 0.594 |

9F. All enrolled subjects; results were achieved with adjustment of
gestational age at blood sample collection.

| | | 95CI | | |
|---|---|---|---|---|
| Characteristics | HR | (L) | 95CI (H) | P value |
| BMI prior to pregnancy (kg/m²) | 1.08 | 1.02 | 1.15 | 0.007 |
| Age (year) | 1 | 0.96 | 1.04 | 0.9325 |
| Concurrent diabetes during pregnancy | 1.06 | 0.58 | 1.94 | 0.8525 |

Table 10. Multivariate hazard ratio analysis of the patient characteristics.

10A. Enrolled subjects blood collected < 34 weeks of gestational age;
results were achieved without adjustment of gestational age at blood
sample collection.

| | | 95CI | | |
|---|---|---|---|---|
| Characteristics | HR | (L) | 95CI (H) | P value |
| BMI prior to pregnancy (kg/m²) | 1.12 | 1.02 | 1.24 | 0.0178 |
| Age (year) | 1.01 | 0.96 | 1.06 | 0.739 |
| Concurrent diabetes during pregnancy | 0.74 | 0.32 | 1.74 | 0.4964 |

10B. Enrolled subjects blood collected ≥ 34 weeks of gestational age;
results were achieved without adjustment of gestational age at blood
sample collection.

| | | 95CI | | |
|---|---|---|---|---|
| Characteristics | HR | (L) | 95CI (H) | P value |
| BMI prior to pregnancy (kg/m²) | 1.09 | 1 | 1.18 | 0.0414 |
| Age (year) | 0.99 | 0.93 | 1.05 | 0.6943 |
| Concurrent diabetes during pregnancy | 1.03 | 0.43 | 2.47 | 0.941 |

10C. All enrolled subjects; results were achieved without
adjustment of gestational age at blood sample collection.

| Characteristics | HR | 95CI (L) | 95CI (H) | P value |
|---|---|---|---|---|
| BMI prior to pregnancy (kg/m²) | 1.08 | 1.02 | 1.14 | 0.0125 |
| Age (year) | 1 | 0.96 | 1.04 | 0.9657 |
| Concurrent diabetes during | 0.92 | 0.5 | 1.68 | 0.7844 |
| pregnancy | | | | |

10D. Enrolled subjects blood collected <34 weeks
of gestational age; results were achieved with adjustment
of gestational age at blood sample collection.

| Characteristics | HR | 95CI (L) | 95CI (H) | P value |
|---|---|---|---|---|
| BMI prior to pregnancy (kg/m²) | 1.13 | 1.03 | 1.24 | 0.0111 |
| Age (year) | 1 | 0.95 | 1.05 | 0.9366 |
| Concurrent diabetes during | 0.74 | 0.32 | 1.74 | 0.4911 |
| pregnancy | | | | |

10E. Enrolled subjects blood collected ≥34 weeks
of gestational age; results were achieved with adjustment
of gestational age at blood sample collection.

| Characteristics | HR | 95CI (L) | 95CI (H) | P value |
|---|---|---|---|---|
| BMI prior to pregnancy (kg/m²) | 1.09 | 1 | 1.19 | 0.0484 |
| Age (year) | 0.99 | 0.93 | 1.06 | 0.7654 |
| Concurrent diabetes during | 1.2 | 0.48 | 3.02 | 0.7005 |
| pregnancy | | | | |

10F. All enrolled subjects; results were achieved with adjustment
of gestational age at blood sample collection.

| Characteristics | HR | 95CI (L) | 95CI (H) | P value |
|---|---|---|---|---|
| BMI prior to pregnancy (kg/m²) | 1.09 | 1.02 | 1.15 | 0.0065 |
| Age (year) | 0.99 | 0.96 | 1.03 | 0.7475 |
| Concurrent diabetes during | 0.97 | 0.53 | 1.79 | 0.9307 |
| pregnancy | | | | |

TABLE 11
Levels of biomarker analyte (ng/ml) detected at different stage of gestation in pregnant
individuals with preeclampsia outcome. Median IRQ and mean SD values are provided.

| | | | <34 weeks | | | | ≥34 weeks | | | |
|---|---|---|---|---|---|---|---|---|---|---|
| | | | Control | | PE | | Control | | PE | |
| | PE trend | | Median | Mean | Median | Mean | Median | Mean | Median | Mean |
| Marker | Early | Late | (IQR) | (SD) | (IQR) | (SD) | (IQR) | (SD) | (IQR) | (SD) |
| Activin | Up | Up | 2.48 | 2.78 | 6.51 | 6.08 | 4.8 | 4.42 | 6.11 | 5.81 |
| A | | | (1.44, 3.72) | (1.59) | (5.67, 7.24) | (1.51) | (3.72, 5.49) | (1.45) | (5.47, 6.45) | (1.22) |
| ENG | Up | Up | 4.44 | 7.99 | 60.57 | 129.55 | 8.75 | 10.43 | 27.8 | 48.68 |
| | | | (3.94, 5.53) | (20.76) | (23.46, 104.37) | (238.95) | (5.53, 12.7) | (7.67) | (13.95, 61.33) | (64.42) |
| EPCR | Up | Down | 4826.16 | 5425.07 | 6405.72 | 7323.87 | 6596.14 | 6890.24 | 6400.14 | 6654.74 |
| | | | (4279.2, | (1786.1) | (5459.1, 7459.49) | (3333.7) | (5374.6, 7658.25) | (2213.42) | (5448.41, 7597.6) | (1825.41) |
| | | | 5924.16) | | | | | | | |
| PIGF | Down | Down | 0.53 | 0.64 | 0.09 | 0.13 | 0.31 | 0.46 | 0.16 | 0.21 |
| | | | (0.38, 0.79) | (0.43) | (0.05, 0.15) | (0.15) | (0.23, 0.5) | (0.38) | (0.1, 0.25) | (0.19) |
| sFlt-1 | Up | Up | 0.49 | 0.93 | 16.3 | 20.29 | 2.7 | 2.75 | 7.64 | 10.72 |
| | | | (0.04, 1.49) | (1.12) | (7.93, 24) | (17.82) | (1.29, 3.55) | (1.72) | (4.43, 13.88) | (9.6) |

Table 12. Univariate odds ratio analysis of the validated markers without adjustment of gestational age at blood sample collection.

12A. Enrolled subjects blood collected
<34 weeks of gestational age.

| Marker | OR | 95CI (L) | 95CI (H) | P value |
|---|---|---|---|---|
| Activin A | 2.73 | 2.05 | 3.88 | <0.001 |
| ENG | 1.08 | 1.04 | 1.12 | <0.001 |
| EPCR | 1 | 1 | 1 | 0.0014 |
| PIGF | 0 | 0 | 0 | <0.001 |
| sFlt-1 | 2.43 | 1.7 | 4.01 | <0.001 |

12B. Enrolled subjects blood collected
≥34 weeks of gestational age.

| Marker | OR | 95CI (L) | 95CI (H) | P value |
|---|---|---|---|---|
| Activin A | 2.31 | 1.54 | 3.78 | <0.001 |
| ENG | 1.12 | 1.06 | 1.2 | <0.001 |
| EPCR | 1 | 1 | 1 | 0.6014 |
| PIGF | 0.01 | 0 | 0.12 | 0.0025 |
| sFlt-1 | 1.57 | 1.28 | 2.05 | <0.001 |

12C. All enrolled subjects.

| Marker | OR | 95CI (L) | 95CI (H) | P value |
|---|---|---|---|---|
| Activin A | 2.5 | 1.99 | 3.27 | <0.001 |
| ENG | 1.09 | 1.06 | 1.13 | <0.001 |
| EPCR | 1 | 1 | 1 | 0.0057 |
| PIGF | 0 | 0 | 0 | <0.001 |
| sFlt-1 | 1.81 | 1.52 | 2.23 | <0.001 |

Table 13. Univariate odds ratio analysis of the validated markers with adjustment of gestational age at blood sample collection.

13A. Enrolled subjects blood collected
<34 weeks of gestational age.

| Marker | OR | 95CI (L) | 95CI (H) | P value |
|---|---|---|---|---|
| Activin A | 3.38 | 2.37 | 5.32 | <0.001 |
| ENG | 1.08 | 1.05 | 1.12 | <0.001 |
| EPCR | 1 | 1 | 1 | 0.0014 |
| PIGF | 0 | 0 | 0 | <0.001 |
| sFlt-1 | 2.58 | 1.76 | 4.4 | <0.001 |

13B. Enrolled subjects blood collected 34 weeks of gestational age.

| Marker | OR | 95CI (L) | 95CI (H) | P value |
|---|---|---|---|---|
| Activin A | 2.36 | 1.57 | 3.9 | <0.001 |
| ENG | 1.11 | 1.06 | 1.2 | <0.001 |
| EPCR | 1 | 1 | 1 | 0.5935 |
| PIGF | 0.01 | 0 | 0.12 | 0.0026 |
| sFlt-1 | 1.58 | 1.28 | 2.06 | <0.001 |

13C. All enrolled subjects.

| Marker | OR | 95CI (L) | 95CI (H) | P value |
|---|---|---|---|---|
| Activin A | 2.83 | 2.2 | 3.81 | <0.001 |
| ENG | 1.09 | 1.06 | 1.13 | <0.001 |
| EPCR | 1 | 1 | 1 | 0.0056 |
| PIGF | 0 | 0 | 0 | <0.001 |
| sFlt-1 | 1.89 | 1.56 | 2.39 | <0.001 |

Table 14. Multivariate odds ratio analysis of the validated markers without adjustment of gestational age at blood sample collection.

14A. Enrolled subjects blood collected
<34 weeks of gestational age.

| Marker | OR | 95CI (L) | 95CI (H) | P value |
|---|---|---|---|---|
| Activin A | 0.95 | 0.51 | 1.7 | 0.8658 |
| ENG | 1 | 0.97 | 1.02 | 0.735 |
| EPCR | 1 | 1 | 1 | 0.0482 |
| PIGF | 0.01 | 0 | 0.27 | 0.0164 |
| sFlt-1 | 2.46 | 1.41 | 5.44 | 0.0074 |

14B. Enrolled subjects blood collected
≥34 weeks of gestational age.

| Marker | OR | 95CI (L) | 95CI (H) | P value |
|---|---|---|---|---|
| Activin A | 1.21 | 0.7 | 2.18 | 0.493 |
| ENG | 1.06 | 1 | 1.15 | 0.0933 |
| EPCR | 1 | 1 | 1 | 0.6009 |
| PIGF | 0.48 | 0.02 | 4.43 | 0.5881 |
| sFlt-1 | 1.2 | 0.94 | 1.72 | 0.2361 |

14C. All enrolled subjects.

| Marker | OR | 95CI (L) | 95CI (H) | P value |
|---|---|---|---|---|
| Activin A | 1.14 | 0.8 | 1.63 | 0.4779 |
| ENG | 1.01 | 0.99 | 1.03 | 0.4367 |
| EPCR | 1 | 1 | 1 | 0.2069 |
| PIGF | 0.06 | 0.01 | 0.48 | 0.0153 |
| sFlt-1 | 1.48 | 1.18 | 1.96 | 0.0031 |

Table 15. Multivariate odds ratio analysis of the validated markers with adjustment of gestational age at blood sample collection.

15A. Enrolled subjects blood collected
<34 weeks of gestational age.

| Marker | OR | 95CI (L) | 95CI (H) | P value |
|---|---|---|---|---|
| Activin A | 1.02 | 0.5 | 2.08 | 0.9597 |
| ENG | 1 | 0.97 | 1.02 | 0.9188 |
| EPCR | 1 | 1 | 1 | 0.0383 |
| PIGF | 0.01 | 0 | 0.36 | 0.0244 |
| sFlt-1 | 2.62 | 1.41 | 6.54 | 0.0109 |

15B. Enrolled subjects blood collected
≥34 weeks of gestational age.

| Marker | OR | 95CI (L) | 95CI (H) | P value |
|---|---|---|---|---|
| Activin A | 1.22 | 0.69 | 2.23 | 0.4953 |
| ENG | 1.06 | 1 | 1.15 | 0.0966 |
| EPCR | 1 | 1 | 1 | 0.6044 |
| PIGF | 0.48 | 0.02 | 4.42 | 0.5859 |
| sFlt-1 | 1.2 | 0.94 | 1.72 | 0.2342 |

15C. All enrolled subjects.

| Marker | OR | 95CI (L) | 95CI (H) | P value |
|---|---|---|---|---|
| Activin A | 1.29 | 0.88 | 1.94 | 0.2022 |
| ENG | 1.01 | 0.99 | 1.03 | 0.4409 |
| EPCR | 1 | 1 | 1 | 0.1811 |
| PIGF | 0.07 | 0.01 | 0.51 | 0.0181 |
| sFlt-1 | 1.48 | 1.17 | 1.98 | 0.0037 |

Table 16. Univariate hazard ratio analysis of the validated markers without adjustment of gestational age at blood sample collection.

16A. Enrolled subjects blood collected
<34 weeks of gestational age.

| Marker | HR | 95CI (L) | 95CI (H) | P value |
|---|---|---|---|---|
| Activin A | 1.65 | 1.42 | 1.91 | <0.001 |
| ENG | 1 | 1 | 1 | <0.001 |
| EPCR | 1 | 1 | 1 | <0.001 |
| PIGF | 0 | 0 | 0.02 | <0.001 |
| sFlt-1 | 1.04 | 1.03 | 1.05 | <0.001 |

16B. Enrolled subjects blood collected 34 weeks of gestational age.

| Marker | HR | 95CI (L) | 95CI (H) | P value |
|---|---|---|---|---|
| Activin A | 2.64 | 1.71 | 4.09 | <0.001 |
| ENG | 1.01 | 1.01 | 1.02 | <0.001 |
| EPCR | 1 | 1 | 1 | 0.5428 |
| PIGF | 0.01 | 0 | 0.16 | <0.001 |
| sFlt-1 | 1.11 | 1.07 | 1.15 | <0.001 |

16C. All enrolled subjects.

| Marker | HR | 95CI (L) | 95CI (H) | P value |
|---|---|---|---|---|
| Activin A | 1.99 | 1.68 | 2.37 | <0.001 |
| ENG | 1 | 1 | 1 | <0.001 |
| EPCR | 1 | 1 | 1 | <0.001 |
| PIGF | 0 | 0 | 0.01 | <0.001 |
| sFlt-1 | 1.05 | 1.04 | 1.06 | <0.001 |

Table 17. Univariate hazard ratio analysis of the validated markers with adjustment of gestational age at blood sample collection.
17A. Enrolled subjects blood collected <34 weeks of gestational age.

| Marker | HR | 95CI (L) | 95CI (H) | P value |
|---|---|---|---|---|
| Activin A | 2.77 | 2.17 | 3.53 | <0.001 |
| ENG | 1 | 1 | 1 | <0.001 |
| EPCR | 1 | 1 | 1 | <0.001 |
| PIGF | 0 | 0 | 0.01 | <0.001 |
| sFlt-1 | 1.04 | 1.03 | 1.05 | <0.001 |

17B. Enrolled subjects blood collected 34 weeks of gestational age.

| Marker | HR | 95CI (L) | 95CI (H) | P value |
|---|---|---|---|---|
| Activin A | 3.14 | 2.11 | 4.67 | <0.001 |
| ENG | 1.01 | 1.01 | 1.02 | <0.001 |
| EPCR | 1 | 1 | 1 | 0.6606 |
| PIGF | 0.01 | 0 | 0.12 | <0.001 |
| sFlt-1 | 1.11 | 1.07 | 1.15 | <0.001 |

17C. All enrolled subjects.

| Marker | HR | 95CI (L) | 95CI (H) | P value |
|---|---|---|---|---|
| Activin A | 3.06 | 2.5 | 3.74 | <0.001 |
| ENG | 1 | 1 | 1 | <0.001 |
| EPCR | 1 | 1 | 1 | <0.001 |
| PIGF | 0 | 0 | 0.01 | <0.001 |
| sFlt-1 | 1.05 | 1.04 | 1.06 | <0.001 |

Table 18. Multivariate hazard ratio analysis of the validated markers without adjustment of gestational age at blood sample collection.
18A. Enrolled subjects blood collected <34 weeks of gestational age.

| Marker | HR | 95CI (L) | 95CI (H) | P value |
|---|---|---|---|---|
| Activin A | 1.27 | 1.07 | 1.51 | 0.0062 |
| ENG | 1 | 1 | 1 | 0.7882 |
| EPCR | 1 | 1 | 1 | 0.5847 |
| PIGF | 0.01 | 0 | 0.1 | <0.001 |
| sFlt-1 | 1 | 0.99 | 1.02 | 0.7271 |

18B. Enrolled subjects blood collected 34 weeks of gestational age.

| Marker | HR | 95CI (L) | 95CI (H) | P value |
|---|---|---|---|---|
| Activin A | 1.84 | 1.17 | 2.9 | 0.0078 |
| ENG | 1 | 1 | 1.01 | 0.2104 |
| EPCR | 1 | 1 | 1 | 0.4431 |
| PIGF | 0.26 | 0.02 | 3.01 | 0.2837 |
| sFlt-1 | 1.03 | 0.98 | 1.09 | 0.228 |

18C. All enrolled subjects.

| Marker | HR | 95CI (L) | 95CI (H) | P value |
|---|---|---|---|---|
| Activin A | 1.43 | 1.19 | 1.71 | <0.001 |
| ENG | 1 | 1 | 1 | 0.3422 |
| EPCR | 1 | 1 | 1 | 0.1955 |
| PIGF | 0.02 | 0 | 0.13 | <0.001 |
| sFlt-1 | 1.02 | 1 | 1.03 | 0.0514 |

Table 19. Multivariate hazard ratio analysis of the validated markers with adjustment of gestational age at blood sample collection.
19A. Enrolled subjects blood collected <34 weeks of gestational age.

| Marker | HR | 95CI (L) | 95CI (H) | P value |
|---|---|---|---|---|
| Activin A | 2.16 | 1.62 | 2.87 | <0.001 |
| ENG | 1 | 1 | 1 | 0.925 |
| EPCR | 1 | 1 | 1 | 0.3497 |
| PIGF | 0.01 | 0 | 0.13 | <0.001 |
| sFlt-1 | 0.99 | 0.97 | 1.01 | 0.4795 |

19B. Enrolled subjects blood collected 34 weeks of gestational age.

| Marker | HR | 95CI (L) | 95CI (H) | P value |
|---|---|---|---|---|
| Activin A | 2.37 | 1.53 | 3.7 | <0.001 |
| ENG | 1 | 1 | 1.01 | 0.4855 |
| EPCR | 1 | 1 | 1 | 0.6818 |
| PIGF | 0.14 | 0.01 | 1.72 | 0.1238 |
| sFlt-1 | 1.02 | 0.97 | 1.07 | 0.4191 |

19C. All enrolled subjects.

| Marker | HR | 95CI (L) | 95CI (H) | P value |
|---|---|---|---|---|
| Activin A | 2.53 | 2 | 3.2 | <0.001 |
| ENG | 1 | 1 | 1 | 0.8254 |
| EPCR | 1 | 1 | 1 | 0.1199 |
| PIGF | 0.02 | 0 | 0.11 | <0.001 |
| sFlt-1 | 0.99 | 0.98 | 1.01 | 0.5504 |

TABLE 20
Different panels of biomarker combinations.

| Marker | Panel 1 | Panel 2 | Panel 3 | Panel 4 | Panel 5 | Panel 6 | Panel 7 |
|---|---|---|---|---|---|---|---|
| *Activin A | + | − | + | − | + | − | + |
| *ENG | − | + | + | − | − | + | + |
| *EPCR | − | − | − | + | + | + | + |
| *sFlt-1 | − | − | − | − | − | − | − |
| ^{Δ}PIGF | + | + | + | + | + | + | + |

| Marker | Panel 8 | Panel 9 | Panel 10 | Panel 11 | Panel 12 | Panel 13 | Panel 14 | Panel 15 |
|---|---|---|---|---|---|---|---|---|
| *Activin A | − | + | − | + | − | + | − | + |
| *ENG | − | − | + | + | − | − | + | + |
| *EPCR | − | − | − | − | + | + | + | + |
| sFlt-1 | + | + | + | + | + | + | + | + |
| ^{Δ}PIGF | + | + | + | + | + | + | + | + |

*Biomarker concentration median goes up from control to case.
^{Δ}Biomarker concentration median goes down from control to case.

TABLE 21
ROC AUC and p value of each biomarker panel at different gestational ages. Scores of each
panel were calculated using the geometric mean of the concentration of each biomarker in that panel.

| | <34 weeks | | ≥34 weeks | | Overall ROC | |
|---|---|---|---|---|---|---|
| Panel | ROC AUC | P value | ROC AUC | P value | AUC | P value |
| Activin A/PIGF | 0.9708 | 2.95E−19 | 0.8531 | 3.66E−09 | 0.9397 | 3.22E−27 |
| ENG/PIGF | 0.9589 | 2.2E−18 | 0.8406 | 1.47E−08 | 0.9261 | 1.12E−25 |
| Activin A × ENG/PIGF | 0.9717 | 2.56E−19 | 0.8575 | 2.21E−09 | 0.9377 | 5.46E−27 |
| EPCR/PIGF | 0.9425 | 3.18E−17 | 0.7862 | 2.55E−06 | 0.9004 | 6.72E−23 |
| Activin A × EPCR/PIGF | 0.9778 | 8.98E−20 | 0.8369 | 2.19E−08 | 0.9419 | 1.8E−27 |
| ENG × EPCR/PIGF | 0.9694 | 3.74E−19 | 0.8356 | 2.5E−08 | 0.9327 | 2.02E−26 |
| Activin A × ENG × EPCR/ | 0.9736 | 1.84E−19 | 0.8519 | 4.23E−09 | 0.9394 | 3.48E−27 |
| PIGF | | | | | | |
| sFlt-1/PIGF | 0.9581 | 2.52E−18 | 0.8288 | 5.09E−08 | 0.9284 | 6.17E−26 |
| Activin A × sFlt-1/PIGF | 0.9597 | 1.91E−18 | 0.84 | 1.57E−08 | 0.9311 | 3.07E−26 |
| ENG × sFlt-1/PIGF | 0.9528 | 6.01E−18 | 0.835 | 2.67E−08 | 0.9271 | 8.63E−26 |
| Activin A × ENG × sFlt-1/ | 0.9553 | 3.99E−18 | 0.8419 | 1.28E−08 | 0.9282 | 6.5E−26 |
| PIGF | | | | | | |
| EPCR × sFlt-1/PIGF | 0.9617 | 1.38E−18 | 0.8262 | 6.55E−08 | 0.9297 | 4.41E−26 |
| Activin A × EPCR × sFlt-1/ | 0.9628 | 1.15E−18 | 0.8356 | 2.5E−08 | 0.9322 | 2.31E−26 |
| PIGF | | | | | | |
| ENG × EPCR × sFlt-1/ | 0.9572 | 2.89E−18 | 0.8344 | 2.85E−08 | 0.9288 | 5.57E−26 |
| PIGF | | | | | | |
| Activin A × ENG × EPCR × | 0.96 | 1.83E−18 | 0.84 | 1.57E−08 | 0.9297 | 4.41E−26 |
| sFlt-1/PIGF | | | | | | |

TABLE 22
Comparison of biomarker's abundances at
early and late gestational age time points.

| | |
|---|---|
| Control | PE |

| Marker | Fold* | P value** | Fold* | P value** |
|---|---|---|---|---|
| Activin A | 0.516042 | 3.36E−06 | 1.064039 | 0.06682 |
| ENG | 0.507138 | 1.64E−08 | 2.179073 | 0.019312 |
| EPCR | 0.731665 | 2.31E−05 | 1.000871 | 0.832824 |
| PIGF | 1.70086 | 0.002692 | 0.569844 | 0.001082 |
| sFlt-1 | 0.181702 | 1.28E−08 | 2.134909 | 0.002454 |

*Fold was calculated by the ratio of the medians of early (<34 weeks) and late (≥34 weeks) gestational age samples' assayed biomarker abundances.
**P value: Mann-Whitney U test.

TABLE 23
Sensitivity and specificity analyses for each biomarker panels in Table 20.
Sensitivity and specificity were calculated by a threshold for binary
classification that gives the maximum value of the sum of the squares
of sensitivity and specificity.

| | <34 weeks | | ≥34 weeks | | | |
|---|---|---|---|---|---|---|
| | Sen- | | Sen- | | Overall | |
| Panel | sitivity | Specificity | sitivity | Specificity | Sensitivity | Specificity |
| 1 | 0.9333 | 0.95 | 0.65 | 0.975 | 0.77 | 0.97 |
| 2 | 0.9667 | 0.8667 | 0.75 | 0.9 | 0.75 | 0.96 |
| 3 | 0.95 | 0.9333 | 0.625 | 1 | 0.83 | 0.91 |
| 4 | 0.9167 | 0.9167 | 0.4 | 0.975 | 0.8 | 0.87 |
| 5 | 0.9167 | 0.9833 | 0.525 | 0.975 | 0.83 | 0.93 |
| 6 | 0.9 | 0.95 | 0.6 | 0.975 | 0.84 | 0.89 |
| 7 | 0.9167 | 0.95 | 0.625 | 0.975 | 0.87 | 0.88 |
| 8 | 0.9333 | 0.9833 | 0.725 | 0.925 | 0.82 | 0.96 |
| 9 | 0.95 | 0.9833 | 0.65 | 1 | 0.89 | 0.88 |
| 10 | 0.95 | 0.9667 | 0.65 | 1 | 0.89 | 0.86 |
| 11 | 0.95 | 0.95 | 0.675 | 1 | 0.85 | 0.9 |
| 12 | 0.95 | 0.9667 | 0.575 | 1 | 0.78 | 0.97 |
| 13 | 0.95 | 0.9667 | 0.625 | 1 | 0.81 | 0.96 |
| 14 | 0.9333 | 0.9667 | 0.625 | 1 | 0.86 | 0.89 |
| 15 | 0.9333 | 0.9667 | 0.625 | 1 | 0.88 | 0.89 |

Table 24. Sensitivity with given specificity levels for each biomarker panels shown in Table 20. Specificity levels were chosen of 1, 0.95, and 0.85 for early stage PE onset (<34 weeks gestation), and of 1, 0.95, 0.9, 0.85, and 0.8 for late stage PE onset 34 weeks gestation), and overall summary.

24A. Enrolled subjects blood collected <34 weeks of gestational age.

| Spe- | | | | | | | | | | | | | | | |
|---|---|---|---|---|---|---|---|---|---|---|---|---|---|---|---|
| cificity | | | | | | | | | Panel | Panel | Panel | Panel | Panel | Panel | Panel |
| level | Panel 1 | Panel 2 | Panel 3 | Panel 4 | Panel 5 | Panel 6 | Panel 7 | Panel 8 | 9 | 10 | 11 | 12 | 13 | 14 | 15 |
| 1 | 0.7333 | 0.45 | 0.7 | 0.2 | 0.8167 | 0.5 | 0.7167 | 0.8667 | 0.8 | 0.7167 | 0.7667 | 0.8667 | 0.8667 | 0.7667 | 0.7833 |
| 0.95 | 0.9333 | 0.8 | 0.8833 | 0.6833 | 0.9167 | 0.9 | 0.9167 | 0.95 | 0.95 | 0.95 | 0.95 | 0.95 | 0.95 | 0.95 | 0.95 |
| 0.85 | 0.95 | 0.9667 | 0.9833 | 0.9333 | 0.9667 | 0.9667 | 0.9667 | 0.95 | 0.95 | 0.95 | 0.95 | 0.95 | 0.95 | 0.95 | 0.95 |

24B. Enrolled subjects blood collected ≥34 weeks of gestational age.

| Spe- | | | | | | | | | | | | | | | |
|---|---|---|---|---|---|---|---|---|---|---|---|---|---|---|---|
| cificity | | | | | | | | | Panel | Panel | Panel | Panel | Panel | Panel | Panel |
| level | Panel 1 | Panel 2 | Panel 3 | Panel 4 | Panel 5 | Panel 6 | Panel 7 | Panel 8 | 9 | 10 | 11 | 12 | 13 | 14 | 15 |
| 1 | 0.45 | 0.575 | 0.625 | 0.125 | 0.475 | 0.55 | 0.525 | 0.6 | 0.65 | 0.65 | 0.675 | 0.575 | 0.625 | 0.625 | 0.625 |
| 0.95 | 0.65 | 0.675 | 0.675 | 0.4 | 0.525 | 0.6 | 0.65 | 0.625 | 0.7 | 0.65 | 0.675 | 0.625 | 0.625 | 0.625 | 0.675 |
| 0.9 | 0.65 | 0.75 | 0.725 | 0.45 | 0.6 | 0.675 | 0.65 | 0.725 | 0.7 | 0.675 | 0.7 | 0.675 | 0.725 | 0.675 | 0.675 |
| 0.85 | 0.7 | 0.75 | 0.75 | 0.6 | 0.7 | 0.675 | 0.7 | 0.725 | 0.75 | 0.725 | 0.725 | 0.725 | 0.725 | 0.7 | 0.7 |
| 0.8 | 0.775 | 0.75 | 0.8 | 0.625 | 0.7 | 0.75 | 0.725 | 0.75 | 0.75 | 0.75 | 0.75 | 0.75 | 0.75 | 0.725 | 0.725 |

24C. All enrolled subjects.

| Specificity | | | | | | | | | Panel | Panel | Panel | Panel | Panel | Panel | Panel |
|---|---|---|---|---|---|---|---|---|---|---|---|---|---|---|---|
| level | Panel 1 | Panel 2 | Panel 3 | Panel 4 | Panel 5 | Panel 6 | Panel 7 | Panel 8 | 9 | 10 | 11 | 12 | 13 | 14 | 15 |
| 1 | 0.6 | 0.34 | 0.56 | 0.15 | 0.66 | 0.39 | 0.64 | 0.75 | 0.73 | 0.62 | 0.7 | 0.74 | 0.73 | 0.68 | 0.72 |
| 0.95 | 0.77 | 0.76 | 0.75 | 0.57 | 0.77 | 0.75 | 0.73 | 0.82 | 0.79 | 0.73 | 0.75 | 0.79 | 0.81 | 0.73 | 0.74 |
| 0.9 | 0.82 | 0.81 | 0.83 | 0.7 | 0.83 | 0.78 | 0.81 | 0.85 | 0.85 | 0.84 | 0.85 | 0.85 | 0.84 | 0.83 | 0.8 |
| 0.85 | 0.89 | 0.85 | 0.85 | 0.8 | 0.86 | 0.85 | 0.87 | 0.88 | 0.89 | 0.89 | 0.87 | 0.87 | 0.87 | 0.88 | 0.88 |
| 0.8 | 0.9 | 0.86 | 0.87 | 0.86 | 0.89 | 0.87 | 0.89 | 0.89 | 0.89 | 0.89 | 0.87 | 0.89 | 0.9 | 0.88 | 0.88 |

Table 25. Specificity with given sensitivity levels for each biomarker panels in shown Table 20. Sensitivity levels were chosen of 1, 0.95, and 0.85 for early stage PE onset (<34 weeks gestation), and of 1, 0.95, 0.9, 0.85, and 0.8 for late stage PE onset 34 weeks gestation) and overall summary.

25A. Enrolled subjects blood collected <34 weeks of gestational age.

| Sen- | | | | | | | | | | | | | | | |
|---|---|---|---|---|---|---|---|---|---|---|---|---|---|---|---|
| sitivity | | | | | | | | Panel | | Panel | Panel | Panel | Panel | Panel | Panel |
| level | Panel 1 | Panel 2 | Panel 3 | Panel 4 | Panel 5 | Panel 6 | Panel 7 | 8 | Panel 9 | 10 | 11 | 12 | 13 | 14 | 15 |
| 1 | 0.15 | 0.1333 | 0.15 | 0.3667 | 0.2667 | 0.35 | 0.25 | 0.15 | 0.1333 | 0.1 | 0.1167 | 0.2 | 0.1667 | 0.2 | 0.1667 |
| 0.95 | 0.9333 | 0.8833 | 0.9333 | 0.8333 | 0.8833 | 0.8667 | 0.9167 | 0.95 | 0.9833 | 0.9667 | 0.95 | 0.9667 | 0.9667 | 0.95 | 0.95 |
| 0.85 | 0.9667 | 0.9167 | 0.9667 | 0.9167 | 0.9833 | 0.95 | 0.9667 | 1 | 0.9833 | 0.9667 | 0.9667 | 1 | 1 | 0.9667 | 0.9667 |

25B. Enrolled subjects blood collected ≥34 weeks of gestational age.

| Sensitivity | | | | | | | | Panel | | Panel | Panel | Panel | Panel | Panel | Panel |
|---|---|---|---|---|---|---|---|---|---|---|---|---|---|---|---|
| level | Panel 1 | Panel 2 | Panel 3 | Panel 4 | Panel 5 | Panel 6 | Panel 7 | 8 | Panel 9 | 10 | 11 | 12 | 13 | 14 | 15 |
| 1 | 0.2 | 0.2 | 0.225 | 0.2 | 0.225 | 0.25 | 0.225 | 0.05 | 0.05 | 0.1 | 0.175 | 0.025 | 0.025 | 0.15 | 0.175 |
| 0.95 | 0.25 | 0.2 | 0.325 | 0.3 | 0.3 | 0.35 | 0.35 | 0.1 | 0.225 | 0.25 | 0.2 | 0.225 | 0.25 | 0.225 | 0.225 |
| 0.9 | 0.5 | 0.35 | 0.45 | 0.475 | 0.4 | 0.35 | 0.45 | 0.325 | 0.275 | 0.3 | 0.375 | 0.35 | 0.275 | 0.35 | 0.35 |
| 0.85 | 0.575 | 0.45 | 0.525 | 0.525 | 0.6 | 0.475 | 0.55 | 0.45 | 0.525 | 0.45 | 0.5 | 0.4 | 0.525 | 0.475 | 0.5 |
| 0.8 | 0.775 | 0.725 | 0.8 | 0.575 | 0.65 | 0.675 | 0.775 | 0.725 | 0.725 | 0.7 | 0.75 | 0.725 | 0.75 | 0.725 | 0.775 |

25C. All enrolled subjects.

| Sensitivity | | | | | | | | Panel | | Panel | Panel | Panel | Panel | Panel | Panel |
|---|---|---|---|---|---|---|---|---|---|---|---|---|---|---|---|
| level | Panel 1 | Panel 2 | Panel 3 | Panel 4 | Panel 5 | Panel 6 | Panel 7 | 8 | Panel 9 | 10 | 11 | 12 | 13 | 14 | 15 |
| 1 | 0.11 | 0.1 | 0.1 | 0.27 | 0.18 | 0.24 | 0.16 | 0.09 | 0.08 | 0.06 | 0.07 | 0.12 | 0.1 | 0.12 | 0.1 |
| 0.95 | 0.62 | 0.61 | 0.67 | 0.6 | 0.69 | 0.65 | 0.66 | 0.42 | 0.53 | 0.55 | 0.53 | 0.55 | 0.58 | 0.55 | 0.56 |
| 0.9 | 0.81 | 0.71 | 0.77 | 0.73 | 0.78 | 0.71 | 0.77 | 0.74 | 0.79 | 0.76 | 0.77 | 0.78 | 0.81 | 0.77 | 0.77 |
| 0.85 | 0.89 | 0.85 | 0.88 | 0.81 | 0.86 | 0.87 | 0.88 | 0.9 | 0.9 | 0.89 | 0.9 | 0.9 | 0.89 | 0.89 | 0.89 |
| 0.8 | 0.93 | 0.91 | 0.92 | 0.87 | 0.93 | 0.89 | 0.9 | 0.96 | 0.93 | 0.92 | 0.92 | 0.94 | 0.96 | 0.9 | 0.9 |

TABLE 26
Importance of each marker in a panel
using random forests modeling.

| | Importance | | |
|---|---|---|---|
| Marker | <34 weeks | ≥34 weeks | Overall |
| Activin A | 8.49 | 5.46 | 10.31 |
| ENG | 10.62 | 12.83 | 13.62 |
| EPCR | 7.85 | −0.84 | 8.76 |
| PIGF | 14.96 | 5.60 | 14.68 |
| sFlt-1 | 23.20 | 10.84 | 20.02 |

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method of providing a preeclampsia marker level representation for a subject, the method comprising:
evaluating a panel of preeclampsia markers in a blood sample from a subject to determine the level of each preeclampsia marker in the blood sample; and
obtaining the preeclampsia marker level representation based on the level of each preeclampsia marker in the panel,
wherein the panel of preeclampsia markers comprise inhibin beta A (Activin A).

2. The method according to claim 1, wherein the panel of preeclampsia markers further comprise one or more preeclampsia markers selected from the group consisting of endoglin (ENG), endothelial protein C receptor (EPCR), soluble fms-like tyrosine kinase-1 (sFlt-1), and placenta growth factor (PlGF).

3. The method of claim 1, wherein the measurement is performed for no more than seven markers for the subject.

4. The method of claim 1, wherein the measurement is performed for no more than five markers for the subject.

5. The method of claim 1, wherein the method does not include measuring the expression levels of any of ADAM12 or PAPPA2.

6. The method of claim 1, wherein the method does not include measuring the expression levels of any of FSTL3, APLN, LEP, INHA, PIK3CB, SLC2A1, CRH, HSD17B1, SIGLEC6, PVRL4, HEXB, IL1RAP, MFAP5, HTRA1, EBI3, HTRA4.

7. The method according to claim 2, wherein the panel of preeclampsia markers comprises inhibin beta A (Activin A), endoglin (ENG), endothelial protein C receptor (EPCR), and placenta growth factor (PlGF).

8. The method according to claim 2, wherein the panel of preeclampsia markers comprises inhibin beta A (Activin A) and placenta growth factor (PlGF).

9. The method according to claim 2, wherein the panel of preeclampsia markers consists of inhibin beta A (Activin A) and placenta growth factor (PlGF).

10. The method according to claim 1, further comprising providing a report of the preeclampsia marker level representation.

11. The method according to claim 1, wherein the preeclampsia marker representation is a preeclampsia score.

12. The method according to claim 9, wherein the preeclampsia marker representation is a ratio of Activin A/PlGF.

13. A method for providing a preeclampsia diagnosis for a subject, the method comprising:
obtaining a preeclampsia marker level representation for a sample from a subject, and
providing a preeclampsia diagnosis for the subject based on the preeclampsia marker level representation,
wherein the preeclampsia marker level representation is obtained based on a level of each preeclampsia marker in a panel of preeclampsia markers comprising inhibin beta A (Activin A).

14. The method according to claim 13, wherein the preeclampsia marker level representation is based on the level of preeclampsia markers in the panel of preeclampsia markers further comprising one or more markers selected from the group consisting of endoglin (ENG), endothelial protein C receptor (EPCR), soluble fms-like tyrosine kinase-1 (sFlt-1), and placenta growth factor (PlGF).

15-21. (canceled)

22. The method according to claim 14, wherein the sample is collected at 16-33 weeks of gestation.

23. The method according to claim 14, wherein the sample is collected at 34 or more weeks of gestation.

24. (canceled)

25. The method of claim 14, wherein the subject:
(a) has history of preeclampsia;
(b) is older than 40;
(c) has babies less than two years or more than 10 years apart;
(d) has obesity; or
(e) has history of certain conditions including chronic high blood pressure, migraine headaches, type 1 or type 2 diabetes, kidney disease, a tendency to develop blood clots, or lupus.

26. The method of claim 14, further comprising administering to the subject identified as preeclampsia a procedure to ameliorate the preeclampsia.

27. The method of claim 26, wherein the procedure is selected from the group consisting of medications to lower blood pressure, use of corticosteroids, anticonvulsant medication such as magnesium sulfate, bed rest, and consideration of delivery if the diagnosis was made at or after 37 gestational weeks.

28. A method of treating a pregnant woman as at preeclampsia, comprising:
(a) measuring with antibodies, in a serum sample obtained from the woman, the expression levels of markers in a panel of preeclampsia markers comprising Activin A (inhibin beta A or INHBA); and
(b) administering to the woman a procedure to ameliorate the preeclampsia when the woman is identified as preeclampsia.

29-44. (canceled)
